SEQUENCE LISTING (31 sequences)

SEQ ID NO: 1 - DNA, 1144 bp, Fragaria vesca
atggcaagaatgtcggaacctctagctgttggaagagtcataggagatgttcttgattccttcacccccactacaaaaatgattgtctcttacaacagcaagctcgtctgcaatggacatgagctcttcccttctgcagtcaccgccaaacctagagttgagattcaaggaggcgacatgagatcattcttcactctggtatacatcattttctttctttcctcctttttccgccttttgttttatatcagatctaccagatcgctaactttagttattttcacaggtaatgacagacccagatgttcctggccctagtgatccttatttgaaagagcacctgcactggtatatatcttacagttaatactgtatattatcaaacctagaaacgaacaaacaaagacaataacaattcagataattaatgacttcaatgttcaaactagatatatatgttgtcatattctagaaatttgcagtattgaccaaacaaattgtccaggttaatttatagatcaattgcactcctgcttgtaatttccagattcaaagaaaagattagttatattaattatcgttaatttattctttctcctttacatggtcattcacgtatatattcagaattgtaatcagaaaatctttggatctggaaataaaagcaagtaaaaattacatattatgtcacgaactcttaactagaaaaactagcaaggtataattgtggagtatatacatgccaataagcttggatctaacagttttgccatgaaattgaactatacaggattgtgacagacattcctggcaccacagatgctacatttggtaggttaattcaaactagttcatgagctaccagtacgttatacagctagtgatgatcagcactgatattgtgtatatactacattaattaacaggaagagaagtggtgagctacgagatgccaaggccaaacataggcatccacaggtttgtgtttgttctcttcatgcaaaaacgaaggcagtcggtgaacccgccttcctcaagggatcacttcaacacccgaaccttcgcagccgaaaacgaccttggtgttcctgttgctgccgtttacttcaatgcacagagagaaacggcagcaagaagacgctag

SEQ ID NO: 2 - DNA, 519 bp, Fragaria vesca
atggcaagaatgtcggaacctctagctgttggaagagtcataggagatgttcttgattccttcacccccactacaaaaatgattgtctcttacaacagcaagctcgtctgcaatggacatgagctcttcccttctgcagtcaccgccaaacctagagttgagattcaaggaggcgacatgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacataggcatccacaggtttgtgtttgttctcttcatgcaaaaacgaaggcagtcggtgaacccgccttcctcaagggatcacttcaacacccgaaccttcgcagccgaaaacgaccttggtgttcctgttgctgccgtttacttcaatgcacagagagaaacggcagcaagaagacgctag

SEQ ID NO: 3 - protein, 172 aa, Fragaria vesca
MARMSEPLAVGRVIGDVLDSFTPTTKMIVSYNSKLVCNGHELFPSAVTAKPRVEIQGGDMRSFFTLVMTDPDVPGPSDPYLKEHLHWIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 4 - DNA, 1138 bp, Fragaria x ananassa
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcactgccaaaccgagagttgagattcaaggaggcgacatgagatcattcttcactctggtatttctttctggcctccttttcggccgtttgttttttttatcagatctaccagatcgctaactttatttattttcacaggtaatgacagacccagatgttcctggccctagtgatccttatttgaaagagcacctgcactggtatatcttacagttaatactgtctatatattatcaaacctagaaacgaagaaacaaagacaataactattcagataattattaataacttcaatgttcaaactagatatatatgttgtcatattctagaaatttgcagtattgaccgaacaaattgtccaggttaatttatagatcaattgcgctcctgcttgtaatttccagattcaaagaaaagattagttattttaattattgttaatttattctttctcctttacatggtcattcatgtattcagaattgtaatccgaaaatctttggatctggaaataaaagcaagtaaaaattacatattatgtcacgaactcttaactagaaaaactagcaagttataattgaggagtatagacatgccaataagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtgtatatactacattaattaacaggaagagaagtggtgagctacgagatgccaaggccaaacataggcatccacaggtttgtgtttgttctcttcatgcaaaaacgaaggcagtcggtgaacccgccttcctcaagggatcacttcaacacccgaaccttcgcagccgaaaacgaccttggtgttcctgttgcagccgtttacttcaatgcacagagagaaacggcagcaagaagacgctag

SEQ ID NO: 5 - DNA, 519 bp, Fragaria x ananassa
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcactgccaaaccgagagttgagattcaaggaggcgacatgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacataggcatccacaggtttgtgtttgttctcttcatgcaaaaacgaaggcagtcggtgaacccgccttcctcaagggatcacttcaacacccgaaccttcgcagccgaaaacgaccttggtgttcctgttgcagccgtttacttcaatgcacagagagaaacggcagcaagaagacgctag

SEQ ID NO: 6 - protein, 172 aa, Fragaria x ananassa
MARMSEPLAVGRVIGDVLDSFTPTTKMIVTYNTKLVCNGHELFPSAVTAKPRVEIQGGDMRSFFTLVMTDPDVPGPSDPYLKEHLHWIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 7 - DNA, 804 bp, Fragaria x ananassa
atggcaagaatgtcggaacctctagctgttggaagagtcataggagatgttcttgattccttcacccccactacaaaaatgattgtctcttacaacagcaagctcgtctgcaatggacatgagctcttcccttctgcagtcaccgccaaacctagagttgagattcaaggaggcgacatgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacataggcatccacaggtttgtgtttgttctcttcatgcaaaaacgaaggcagtcggtgaacccgccttcctcaagggatcacttcaacacccgaaccttcgcagccgaaaacgaccttggtgttcctgttgctgccgtttacttcaatgcacagagagaaacggcagcaagaagacgctagctagctaggcaccttgcagggaggcaggccagatcccaaaagatctcttgctataaccagaattaatcctccctaattaaataaaagagaagtgtgtagtgatgaattaatcctccctttggctaggttattaggctaaaattactcgcatcttaccagaataattttcccaaacggagctgtacgtgagtgtttgtgtgactgtcattagcttttctttttcctctctccggcctggaatgttatgaagtaaatttgttcttagtcaaaaaaaaaaaaaaaaaa

SEQ ID NO: 8 - protein, 172 aa, Fragaria x ananassa; identical to SEQ ID NO: 3

SEQ ID NO: 9 - DNA, 577 bp, Fragaria x ananassa
aaaggatatatatagaattctgtatatcaaattccatggcaaagatctcagatcctcttgttgttggaagagtcattggagatgttattgattgtttcacacccagtgttaaaatgacagtgacttacaactccagcaagaaggtgtataatgggcatgagctatatccttcctctgtaaccgtcaagcctaaggttgaagttcatggaggcgatatgagatctttctttacactggtcatgactgacccagatgttccaggtcctagtgatccttacctgaaggagcacttgcactggatagtcacggacatcccaggaacaactgacaacacatttggaagggaggtggtgaaatatgaaatgccgaggccaaacatagggatccacaggtttgtgttccttttgttcaagcagaaaggtaggcagacagtgattcctcctccttcaaaggaccatttcaacagtcgaaagttcgcagaagcaaacgagttcgggcttccggtggctgctgtcttcttcaatgcccagagggaaactgctgcaagaagacgctaaataacttgttaaattaacca

SEQ ID NO: 10 - protein, 173 aa, Fragaria x ananassa
MAKISDPLVVGRVIGDVIDCFTPSVKMTVTYNSSKKVYNGHELYPSSVTVKPKVEVHGGDMRSFFTLVMTDPDVPGPSDPYLKEHLHWIVTDIPGTTDNTFGREVVKYEMPRPNIGIHRFVFLLFKQKGRQTVIPPPSKDHFNSRKFAEANEFGLPVAAVFFNAQRETAARRR

SEQ ID NO: 11 - DNA, 532 bp, Fragaria x ananassa
attgatgtcaaggataatggagaccctgacccttggaagagtggtaggagaggttgtggacatgttcaccccatctgtgaaaatgaatgtagtttacagtagctgcaacaaacaagtatctaatggccacgagatcatgccttctgtcattgctgctaagcctcgcgtcgacatcggtggtgaagatatgagggctgcttatacattgattatgacagaccctgattacccaagccccagtgatccatgcttgagagaacatctccactggatggtgacagacattcctggtaccactgatgtctcctttggaagagaagttgtggagtatgaaactccaaagccggtggtggggattcaccggtacgtgtttctgttgttccagcagactagaggaaggcagacggtgagagttccggcttcgcgggacaacttcaacacaagaaagttttcacaagagaatagtcttggcctgcctgtggctgctgtctacttcaatgcacaaagagaaactgcagccagaagaagatga

SEQ ID NO: 12 - protein, 175 aa, Fragaria x ananassa
MSRIMETLTLGRVVGEVVDMFTPSVKMNVVYSSCNKQVSNGHEIMPSVIAAKPRVDIGGEDMRAAYTLIMTDPDYPSPSDPCLREHLHWMVTDIPGTTDVSFGREVVEYETPKPVVGIHRYVFLLFQQTRGRQTVRVPASRDNFNTRKFSQENSLGLPVAAVYFNAQRETAARRR

SEQ ID NO: 13 - DNA, 2304 bp, Artificial Sequence
  Synthetic
tggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagatctgcaggtcgactctagaatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaaccaataggatccccgggtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgatccccgggtaccaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaac

SEQ ID NO: 14 - DNA, 4037 bp, Artificial Sequence
  Synthetic
tggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgatcgagatctgcaggtcgactctagaatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaaccaataggatccccgggtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgatccccgggtaccatctcgagtgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggacggatccctagcgtcttcttgctgccgtttctctctgtgcattgaagtaaacggctgcaacaggaacaccaaggtcgttttcggctgcgaaggttcgggtgttgaagtgatcccttgaggaaggcgggttcaccgactgccttcgtttttgcatgaagagaacaaacacaaacctgtggatgcctatgtttggccttggcatctcgtagctcaccacttctcttccaaatgtagcatctgtggtgccaggaatgtctgtcacaatccagtgcaggtgctctttcaaataaggatcactagggccaggaacatctgggtctgtcattaccagagtgaagaagcaagaatgtcggaacctctagctgttggaagagtcataggagatgttcttgattccttcaccccaactacaaaaatgattgtcacttacaacaccaagctcgtctgcaatggacatgagctcttcccttctgcagtcactgccaaaccgagagttgagattcaaggaggcgacatgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacataggcatccacaggtttgtgtttgttctcttcatgcaaaaacgaaggcagtcggtgaacccgccttcctcaagggatcacttcaacacccgaaccttcgcagccgaaaacgaccttggtgttcctgttgcagccgtttacttcaatgcacagagagaaacggcagcaagaagacgctaggggcccatggtaccaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaac

SEQ ID NO: 15 - DNA, 20 bp, Artificial Sequence
  Synthetic
caaccacgtcttcaaagcaa

SEQ ID NO: 16 - DNA, 20 bp, Artificial Sequence
  synthetic
catgtcgcctccttgaatct

SEQ ID NO: 17 - DNA, 20 bp, Artificial Sequence
  synthetic
cgcacctaccaaaagcatct

SEQ ID NO: 18 - DNA, 20 bp, Artificial Sequence
  synthetic
caatagcagccagtcccttc

SEQ ID NO: 19 - DNA, 333 bp, Artificial Sequence
  Synthetic
ctagcgtcttcttgctgccgtttctctctgtgcattgaagtaaacggctgcaacaggaacaccaaggtcgttttcggctgcgaaggttcgggtgttgaagtgatcccttgaggaaggcgggttcaccgactgccttcgtttttgcatgaagagaacaaacacaaacctgtggatgcctatgtttggccttggcatctcgtagctcaccacttctcttccaaatgtagcatctgtggtgccaggaatgtctgtcacaatccagtgcaggtgctctttcaaataaggatcactagggccaggaacatctgggtctgtcattaccagagtgaagaa

SEQ ID NO: 20 - DNA, 183 bp, Artificial Sequence
  Synthetic
gcaagaatgtcggaacctctagctgttggaagagtcataggagatgttcttgattccttcaccccaactacaaaaatgattgtcacttacaacaccaagctcgtctgcaatggacatgagctcttcccttctgcagtcactgccaaaccgagagttgagattcaaggaggcgacatgagatca

SEQ ID NO: 21 - DNA, 333 bp, Artificial Sequence
  Synthetic
ttcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacataggcatccacaggtttgtgtttgttctcttcatgcaaaaacgaaggcagtcggtgaacccgccttcctcaagggatcacttcaacacccgaaccttcgcagccgaaaacgaccttggtgttcctgttgcagccgtttacttcaatgcacagagagaaacggcagcaagaagacgctag

SEQ ID NO: 22 - DNA, 531 bp, Fragaria vesca
atgcctagggacagggaccccctcgttgtgggaagagtcataggtgatgttctggacccttttacaaagtctgtttctctcagggtgacttacacttctaaggaggtcaacaatggttgtgagctcaaaccttcccaagttgtcagccaacctcgagttgatataggaggggaggatcttaggaccttctacactctggtcatggtcgatcctgatgcacccagcccaagtgatccccacctgaaagaatatttgcattggttagtcactgatattcctgcaacagctggggcagttttcggccaagagattgtgtgttatgaaagtccacggccaacagcggggattcatcgcttcctttttgtgttgtttcggcagttgggaaggcaaactgtgtatgctccgggatggcgccaaaactttaacaccagagactttgccgagctctacaatcttggatcaccggtggctgccgtctactttaactgccagagagaaagtggctccggcggaaggagaagatcatcgtaa

SEQ ID NO: 23 - protein, 176 aa, Fragaria vesca
MPRDRDPLVVGRVIGDVLDPFTKSVSLRVTYTSKEVNNGCELKPSQVVSQPRVDIGGEDLRTFYTLVMVDPDAPSPSDPHLKEYLHWLVTDIPATAGAVFGQEIVCYESPRPTAGIHRFLFVLFRQLGRQTVYAPGWRQNFNTRDFAELYNLGSPVAAVYFNCQRESGSGGRRRSS

SEQ ID NO: 24 - DNA, 548 bp, Fragaria vesca
ggatccatgcctagggacagggaccccctcgttgtgggaagagtcataggtgatgttctggacccttttacaaagtctgtttctctcagggtgacttacacttctaaggaggtcaacaatggttgtgagctcaaaccttcccaagttgtcagccaacctcgagttgatataggaggggaggatcttaggaccttctacactctggtcatggtcgatcctgatgcacccagcccaagtgatccccacctgaaagaatatttgcattggttagtcactgatattcctgcaacagctggggcagttttcggccaagagattgtgtgttatgaaagtccacggccaacagcggggattcatcgcttcctttttgtgttgtttcggcagttgggaaggcaaactgtgtatgctccgggatggcgccaaaactttaacaccagagactttgccgagctctacaatcttggatcaccggtggctgccgtctactttaactgccagagagaaagtggctccggcggaaggagaagatcatcgtaactagtgggccc

SEQ ID NO: 25 - DNA, 557 bp, Fragaria x ananassa
ccagctagctagcttgaaggatcaatatgcctagggacagggaccccctcgttgtaggaagagtcataggtgatgttctggacccttttacaaagtctgtttctctcagggtgacttacacttctaaggaggtcaacaatggttgtgagctcaaaccttcccaagttgtcagccaacctcgagttgatataggaggggaggatcttaggaccttctacactctggtcatggtcgatcctgatgcacccagcccaagtgatcccaacctgaaagaatatttgcattggttagtcactgatattcctgcaacagctggggcagttttcggccaagagattgtgtgttatgaaagtccacggccaacagcggggattcatcgcttcctttttgtgttgtttcggcagttgggaaggcaaactgtgtatgctccgggatggcgccaaaactttaacaccagagactttgccgagctctacaatcttggatcaccggtggctgccgtctactttaactgccagagagaaagtggctccggcggaaggagaagatcatcgtaa

SEQ ID NO: 26 - protein, 176 aa, Fragaria x ananassa
MPRDRDPLVVGRVIGDVLDPFTKSVSLRVTYTSKEVNNGCELKPSQVVSQPRVDIGGEDLRTFYTLVMVDPDAPSPSDPNLKEYLHWLVTDIPATAGAVFGQEIVCYESPRPTAGIHRFLFVLFRQLGRQTVYAPGWRQNFNTRDFAELYNLGSPVAAVYFNCQRESGSGGRRRSS

SEQ ID NO: 27 - DNA, 586 bp, Fragaria x ananassa
ctactctgaagtagtgagacgttagctagctagctagtttgagtgaaggatcaatatgcctagggacagggaccccctcgttgtgggaagagtcataggtgatgttttggacccttttacaaggtgtgtttctctctgtgtggcttacaattctaaggaggtcaccaatggttgtgaactcaaaccttcccaagttgtcagccatcctcgagttgatataggaggggaggatcttaggaccttctacactctggtcatggtcgatcctgatgcacccagcccaagtgatcccaacctgaaagaatatttgcattggttagtcactgatattcctgcaacagctggggcagttttcggccgagagattgtgtgttatgaaagtccacggccaacagcggggattcatcgcttcctttttgtgttgtttcggcagttgggaaggcaaactgtgtatgctccgggatggcgccaaaactttaacaccagagactttgccgagctctacaatcttggatcaccggtggctgccgtctactttaactgccagagagaaagtggctccggcggaaggagaagatcatcgtaa

SEQ ID NO: 28 - protein, 176 aa, Fragaria x ananassa
MPRDRDPLVVGRVIGDVLDPFTRCVSLCVAYNSKEVTNGCELKPSQVVSHPRVDIGGEDLRTFYTLVMVDPDAPSPSDPNLKEYLHWLVTDIPATAGAVFGREIVCYESPRPTAGIHRFLFVLFRQLGRQTVYAPGWRQNFNTRDFAELYNLGSPVAAVYFNCQRESGSGGRRRSS

SEQ ID NO: 29 - protein, 181 aa, Fragaria x ananassa
MLKSMARARDQEPLVVGRVIGDVIEPFTKSVSLRMTYSNNREVTSGCELKPSHVINRPRVQIGGDDLRNFYTLVMVDPDAPSPSDPNLKEYLHWLVTDIPATTGASFGQEIVSYESPRPSIGIHRFVSVLFRQLGRQTVYAPGWRQNFNTREFAENYNLGSPVAAVFFNCQRESGSGGRRM

SEQ ID NO: 30 - DNA, 725 bp, Fragaria x ananassa
ctgggatgtgttttaattagttgatacttaaatctatggccaaggctagagatcaggatgctcttgtcgtctcgagagtaatcggagatgttatagagccgttcaccaagtctgtgtctttgaggatgacttacattaataaccgggagtttaccaatggctccgagctcaaaccttctcatgttgtccaccgacctcgagttgagataggtggagatgatcttaggaatttctacactctggttatggtagatcctgatgcacccaatccaagtgaacccaacctcaaggaatatttgcactggttggttactgatattccggcaacaactggagcaagcttcggccaagagattgtgagctatgaaagtccacggccagcgatggggatccatcgctttgtttcggttttgtatcgccaattgggtcggaaaacagtttatgctccagaatggagccaaaatttcaacactagaaagtttgctgagaactataatcttggatcaccggtggccgccgtctattttaactgccaaagggaggccggctgtggcggaaggagaactataatgtaatcctgtctattatgttgtctatctaagtctaaccagcactagctagctgtgagttgtgtgatgtaagataaattaattaatgtactagtaattttgtaaggctacttgaatcctgtaggtcgaactttttggaaaaaaaaaaaaaaaaaa

SEQ ID NO: 31 - protein, 179 aa, Fragaria x ananassa
MAKARDQDALVVSRVIGDVIEPFTKSVSLRMTYINNREFTNGSELKPSHVVHRPRVEIGGDDLRNFYTLVMVDPDAPNPSEPNLKEYLHWLVTDIPATTGASFGQEIVSYESPRPAMGIHRFVSVLYRQLGRKTVYAPEWSQNFNTRKFAENYNLGSPVAAVYFNCQREAGCGGRRTIM

(19) United States
(12) Patent Application Publication
(10) Pub. No.: US 2018/0092319 A1
(43) Pub. Date: Apr. 5, 2018

(54) GROWING STRAWBERRY PLUG PLANTS AT LOW ELEVATION WITHOUT THE NEED FOR CONDITIONING

(71) Applicant: J.R. SIMPLOT COMPANY, Boise, ID (US)

(72) Inventors: Rekha CHAWLA, Meridian, ID (US); Michele Lorraine Krucker, Boise, ID (US)

(21) Appl. No.: 15/720,264

(22) Filed: Sep. 29, 2017

Related U.S. Application Data
(60) Provisional application No. 62/402,582, filed on Sep. 30, 2016.

(51) Int. Cl. A01H 5/08; C12N 15/82; A01H 1/06; C07K 14/415

(52) U.S. Cl. CPC A01H 5/08; C12N 15/8218; C07K 14/415; A01H 1/06; C12N 15/8241; C12N 15/8213

(57) ABSTRACT

The present disclosure relates to methods for growing a genetically modified strawberry plant (e.g., a plug plant) having reduced activity of an endogenous TERMINAL FLOWER (TFL) gene. The genetically modified strawberry plants are of the genus Fragaria. Genetically modified strawberry plants with reduced TFL1 activity are grown in a nursery located at any of low elevation, high elevation, northern latitude, southern latitude, or in a controlled environment facility. The yield of strawberry fruit of genetically modified strawberry plants with reduced TFL1 activity is higher than conventionally grown plug plants. The plants of the present disclosure are transformed with the pSIM2441 vector.

### CROSS-REFERENCE TO RELATED APPLICATIONS

The present application claims priority to, and the benefit of U.S. Provisional Patent Application Ser. No. 62/402,582, filed Sep. 30, 2016, which is herein incorporated by reference in its entirety.

### FIELD OF THE INVENTION

The field of the invention concerns growing a genetically modified plug strawberry plant that has reduced activity of a transcription factor encoded by a TERMINAL FLOWER (TFL) gene, resulting in higher yield of strawberry fruit.

### BACKGROUND

Conventional strawberries use environmental cues such as light cycles and temperature to regulate flowering. This process is termed vernalization. In conventional commercial strawberry production, strawberry plug plants are grown in nursery fields for an initial growth season where vernalization occurs. Conventional commercial strawberry production of plug plants typically occurs at (1) a high elevation nursery field, (2) a nursery field located at a more northern latitude for northern hemisphere commercial fruit production, (3) a nursery field located at a more southern latitude for southern hemisphere commercial fruit production, or (4) a controlled environment facility. Plug plants are transported from the nursery fields to different fields for commercial fruit production. In conventional commercial strawberry production, vernalization is necessary for producing a higher total yield of fruit.

Drawbacks of this conventional, commercial strawberry cultivation practice include: 1) exposure of the plants in the nursery field to soil-borne pests and/or pathogens requiring fumigant pesticide; 2) transportation costs of moving plants between nursery and fruit production fields; and 3) increased acreage use for the nursery field and fruit production field.

### SUMMARY

The present disclosure provides compositions and methods for genetically enhancing plant production, such as strawberry plant production. Plants of the present disclosure do not need nursery vernalization step. They can be propagated in sterile soil in greenhouses or screenhouses and can be planted in the fruiting (or commercial production) field at the right age when the weather becomes favorable or in a weather controlled greenhouse year-round.

In some embodiments, plants with one or more modified genes are provided. In some embodiments, the plants belong to the Rosaceae family. In some embodiments, the plants belong to the Fragaria genus. In some embodiments, the plants are strawberry plants.

In some embodiments, the genes are involved in flowering time signaling pathway. In some embodiments, the genes are TFL genes, upstream genes that regulate a TFL gene, or downstream genes that are regulated by TFL genes. In some embodiments, one or more genes in the strawberry plants are modified to bring a blockade or inhibition of TFL, or activation and overexpression of signal transduction cascade downstream thereof that is negatively controlled by TFL. In some embodiments, the TFL gene is TFL1, TFL2, or TFL3. In some embodiments, the TFL1 gene comprises a nucleotide sequence of SEQ ID NO:1, SEQ ID NO:4, SEQ ID NO:2, SEQ ID NO:5, SEQ ID NO:7, or a nucleotide sequence encoding a protein sequence of SEQ ID NO:3, SEQ ID NO:6, or SEQ ID NO:8. In some embodiments, the TFL1 gene comprises a nucleotide sequence that can hybridize to SEQ ID NO:1, SEQ ID NO:4, SEQ ID NO:2, SEQ ID NO:5, SEQ ID NO:7, or a nucleotide sequence encoding a protein sequence of SEQ ID NO:3, SEQ ID NO:6, or SEQ ID NO:8 under stringent hybridization conditions. In some embodiments, the reduced activity of a TFL1 gene is achieved by any suitable method. In some embodiments, the methods include, but are not limited to, reduced gene expression level, reduced gene copy number, reduced gene amplification, reduced RNA activity level, reduced mRNA abundance, reduced mRNA synthesis rate, reduced mRNA stability, reduced protein activity level, reduced protein synthesis, reduced protein abundance, reduced protein stability, reduced protein enzymatic activity, reduced protein phosphorylation, or a combination thereof.

In some embodiments, the reduced activity of a TFL gene (e.g., TFL1 gene) is induced by RNA interference (RNAi), genome editing, or mutation of the endogenous TFL gene. In some embodiments, the RNA interference is induced by expression in a cell of the Fragaria plant an RNAi cassette targeting the endogenous TFL gene, or by topical application of RNAi triggers targeting the endogenous TFL gene. In some embodiments, the genome editing is by expression in a cell of the Fragaria plant a zinc-finger nuclease, a TALE-mediated nuclease, or an RNA-guided nuclease. In some embodiments, the mutation of the endogenous TFL gene is by chemical mutagenesis, radiation mutagenesis, transposon mutagenesis, insertional mutagenesis, signature tagged mutagenesis, site-directed mutagenesis, and/or natural mutagenesis.

In some embodiments, one or more genes in the strawberry plants are modified to bring an activation or overexpression of FT, or of the signal transduction cascade downstream thereof that is positively regulated by FT. In some embodiments, the FT gene is FT1 (e.g., a gene having the DNA sequence—of SEQ ID NO: 22, SEQ ID NO: 24, or SEQ ID NO: 25, or a gene having the DNA sequence encoding a FT1 protein sequence of SEQ ID NO: 23, or SEQ ID NO: 26). In some embodiments, the FT gene is FT2 (DNA sequence SEQ ID NO:27; PRT sequence is SEQ ID NO: 28 or SEQ ID NO: 29). In some embodiments, the FT gene is FT3 (DNA sequence SEQ ID NO: 30; PRT sequence SEQ ID NO: 31). In some embodiments, the FT gene comprises a nucleotide sequence that can hybridize to SEQ ID NO: 22, SEQ ID NO: 24, or SEQ ID NO: 25, or a gene encoding SEQ ID NO: 23 or SEQ ID NO: 26 under stringent hybridization conditions.

The present disclosure also provides methods of growing plants, such as plug plants. In some embodiments, the plug plants are genetically modified plug plants. In some embodiments, the plug plants are of the genus Fragaria. In some embodiments, the plug plants are Fragaria vesca, or Fragaria×ananassa. In some embodiments, the plug plant is a diploid, a tetraploid, a pentaploid, a hexaploid, a octoploid, a decaploid, or has an uncharacterized ploidy. In some embodiments, the plants are strawberry plants. In some embodiments, the plug plant is a June-bearing strawberry plant, an early season June-bearing strawberry plant, an early midseason June-bearing strawberry plant, a midseason June-bearing strawberry plant, a late midseason June-bearing strawberry plant, a late season June-bearing strawberry plant, a short-day strawberry plant variety, a seasonal flowering strawberry plant variety, a long-day strawberry plant variety, a day-neutral strawberry plant variety, a perpetual flowering strawberry plant variety, a recurrent strawberry plant variety, a remontant strawberry plant variety, a long-day strawberry plant variety, or an everbearing strawberry plant variety.

In some embodiments, the methods comprise controlling one or more flowering genes in the plants. In some embodiments, the methods comprise controlling one or more flowering genes in the plants during plant production. In some embodiments, the flowering genes are genes in the TFL signal transduction cascade. In some embodiments, the flowering genes are TFL. In some embodiments, the flowering genes are FT.

Thus, the present disclosure provides methods for growing a genetically modified plug plant. In some embodiments, the genetically modified plug plant has reduced activity of a transcription factor encoded by a TERMINAL FLOWER (TFL) gene compared to a non-genetically modified control plant. The reduced activity of TFL gene in some embodiments encompasses reduced activity of the signal transduction cascade downstream thereof that is positively regulated by TFL. In some embodiments, the genetically modified plug plant has increased activity of Flowering Locus T (FT) compared to a non-genetically modified control plant. The increased activity of TFL gene in some embodiments encompasses increased activity of the signal transduction cascade downstream thereof that is positively regulated by FT.

In some embodiments, the methods comprise growing the plug plant in or on a substrate in a nursery, wherein the genetically modified plug plant flowers independently of temperature or photoperiod.

In some embodiments, the nursery location is (1) a low elevation, (2) a high elevation, (3) a northern latitude for northern hemisphere commercial fruit production, (3) a southern latitude for southern hemisphere commercial fruit production, or (4) in a controlled environment facility.

In some embodiments, the methods do not include conditioning steps traditionally used. Traditionally, strawberry plug plants are grown in a nursery field during the initial growth cycle during which time vernalization occurs. Following this initial growth cycle, the plug plants are moved to a fruiting or production field. During such traditional conditioning, fumigant pesticides are normally used to prevent strawberry plug plants from infections.

Traditionally, strawberry plug plants are grown at high elevations during the initial growth cycle, before being moved to a lower elevation location. During such traditional steps, commercially grown strawberry plug plants are conditioned at an elevation between about 4000-5000 feet above sea level in locations, such as northern California. During such traditional conditioning at high elevations, pesticides are normally used to prevent strawberry plug plants from infections. Conditioning of strawberry plug plants requires the plugs to be grown at high elevations. Typically, commercially grown strawberry plug plants are conditioned at an elevation between 4000-5000 feet above sea level in locations such as northern California. Such high elevations comprise a range of elevations consisting of about 600-1000 feet, about 1000-2000 feet, about 2000-3000 feet, about 3000-4000 feet, about 4000-5000 feet, about 5000-6000 feet, or more above sea level. Methods of the present invention eliminate this step of high elevation conditioning, and do not need to grow plants at such high elevation.

Methods of the present disclosure improve yield of plant, such as the yield of fruit. In some embodiments, the yield of fruit from the genetically modified plants described herein, such as plants with reduced activity of the transcription factor encoded by a TFL gene, or plants with increased activity of an FT gene, is greater than the yield of fruit from non-genetically modified control plants, when grown under the same conditions.

In some embodiments, plants of the methods are grown in substrate that is not treated with a pesticide, such as a fumigant pesticide. In some embodiments, the substrate is soil or earth substrate. In conditioning steps traditionally used, fumigant pesticides are used during strawberry plug plant growth in order to reduce pathogenic infection. In some embodiments, a fumigant pesticides is: methyl bromide, 1,3-dichloropropene, trichloronitromethane, chloropicrin, methyl iodide, tetrahydro-3,5-dimethyl-2 H-1,3,5-thiadiazine-2-thione, sodium N-methyl dithiocarbamate, potassium N-methyl dithiocarbamate, or any combination thereof. Methods of the present application reduce or eliminate the need for fumigant pesticides when compared to plants treated with traditional nursery vernalization step.

A method of the present disclosure reduces the amount of time necessary from the beginning of plug plant growth to the final product of a desired strawberry fruit, by at least about 6 months. As a result, the yield of strawberry plug plants, and subsequent strawberry fruits is increased while growing in the commercial production fields due to the continuous flowering, when compared to plants treated with traditional nursery vernalization step.

Corresponding to the present disclosure, the strawberry plug plants with reduced expression of endogenous a TFL1 gene are able to grow independent of temperature changes and alterations between long and short day cycles, otherwise known as vernalization. Thus, the present disclosure provides a method for producing strawberry plants without the need for high elevation conditioning demonstrated by the non-transformed lines.

In one embodiment, the strawberries produced are of the genus Fragaria. In one embodiment, strawberries are commercially attractive varieties of strawberries that are grown in commercial fields in the United States. In another embodiment, strawberries are commercially attractive varieties of strawberries that are grown in commercial fields outside of the United States. In one embodiment, a commercially attractive variety may be Fragaria×ananassa or Fragaria vesca. In some embodiments, the Fragaria plant is a long-day variety, a short-day variety, or a day neutral variety.

In one embodiment, the method comprises downregulating the TFL signal transduction cascade. Regulatory control of the terminal flowering (TFL) signal transduction cascade prevents the end of flowering in response to environmental cues. In one embodiment, the method comprises downregulating the TFL signal transduction cascade by repression of a TFL1 gene, a TFL2 gene, or a TFL3 gene. In some embodiments, the TFL1 gene has the sequence of SEQ ID NO:1 or SEQ ID NO:4. In another embodiment, the coding region (cDNA) of the TFL1 gene has the sequence of SEQ ID NO:2, SEQ ID NO:5, or SEQ ID NO:7. In another embodiment, the protein sequence of the TFL1 gene has the sequence SEQ ID NO:3, SEQ ID NO:6, or SEQ ID NO:8. In one embodiment, the repression may be RNAi, genome editing tools or any other suitable gene editing tools.

In some embodiments, repression of expression of an endogenous TFL1 gene in a Fragaria plant is done with transformation of a Fragaria plant with a repression vector, such as the pSIM2441 vector. In some embodiments, the pSIM2441 vector comprises the insert region of nucleotide sequence SEQ ID NO: 14. In a further embodiment, the insert region comprises an expression cassette which when expressed in a cell of a transformed Fragaria plant causes repression of an endogenous TFL1 gene. In one embodiment, the RNAi expression cassette FaTFL comprises SEQ ID NO: 19, SEQ ID NO: 20, and SEQ ID NO: 21, operably linked.

In some embodiments, mutations in endogenous a TFL gene is introduced in a cell of a Fragaria plant by a non-natural means such as with chemical mutagenesis, radiation mutagenesis, transposon mutagenesis, insertional mutagenesis, signature tagged mutagenesis, site-directed mutagenesis, and natural mutagenesis.

The present disclosure also provides methods to reduce activity of an endogenous TFL1 gene in a Fragaria plant. In some embodiments, the method comprises transforming the Fragaria plant with the pSIM2441 vector as described herein. In some embodiments, the pSIM2441 vector comprises the insert region of nucleotide sequence SEQ ID NO: 14. In some embodiments, the insert region comprises an expression cassette, wherein the expression cassette comprises on the same strand in operable linkage a sense copy and an antisense copy of a fragment of the TFL1 gene, wherein the sense copy and the antisense copy of the fragment have a length sufficient for gene silencing when expressed in a cell of a transformed Fragaria plant, and wherein the expression cassette comprises SEQ ID NO: 19, SEQ ID NO: 20, and SEQ ID NO: 21, operably linked together.

The present disclosure further provides Fragaria plants or plug plants transformed with the pSIM2441 vector, In some embodiments, the pSIM2441 vector comprises the insert region of nucleotide sequence SEQ ID NO: 14. In some embodiments, the expression cassette when expressed in a cell of the Fragaria plant causes reduced activity of the endogenous TFL1.

### BRIEF DESCRIPTION OF THE DRAWINGS

FIG. 1. Schematic representation of the insert region of vector pSIM2402 between the T-DNA Left Border and Right Border. The insert is 2304 bp in length, as represented by SEQ ID NO: 13.

FIG. 2. Schematic representation of the insert region of vector pSIM2441 between the T-DNA Left Border and Right Border. The insert is 4307 bp in length, as represented by SEQ ID NO: 14.

### DESCRIPTION OF THE TEXT FILE SUBMITTED ELECTRONICALLY

The contents of the text file submitted electronically are incorporated herein by reference in their entirety: A computer readable format copy of the Sequence Listing (filename: JRSI-077-01US_SEQUENCE LISTING_20170914_ST25.txt, date recorded: Sep. 14, 2017, file size 38 kilobytes).

### DETAILED DESCRIPTION

In the description, which follows, a number of terms are used. In order to provide a clear and consistent understanding of the specification and claims, including the scope to be given such terms, the following definitions are provided:

Allele. An allele is any of one or more alternative forms of a gene which relate to one trait or characteristic. In a diploid cell or organism, the two alleles of a given gene occupy corresponding loci on a pair of homologous chromosomes.

Amino acid sequence. As used herein, includes an oligopeptide, peptide, polypeptide, or protein and fragments thereof that are isolated from, native to, or naturally occurring in a plant, or are synthetically made but comprise the nucleic acid sequence of the endogenous counterpart.

“Border” refers to a border sequence derived from an Agrobacterium T-DNA. The T-DNA border promotes and facilitates the integration of a polynucleotide to which it is linked. A DNA insert of the present disclosure preferably contains border sequences (a left border and a right border sequence). A border sequence of a DNA insert is between 5-100 bp in length, 10-80 bp in length, 15-75 bp in length, 15-60 bp in length, 15-50 bp in length, 15-40 bp in length, 15-30 bp in length, 16-30 bp in length, 20-30 bp in length, 21-30 bp in length, 22-30 bp in length, 23-30 bp in length, 24-30 bp in length, 25-30 bp in length, or 26-30 bp in length. A DNA insert left and right border sequences may be derived from Agrobacterium (T-DNA), or the border sequence may be isolated from and/or native to the genome of a plant that is to be modified (P-DNA). A T-DNA border sequence is typically derived from an Agrobacterium species, such as Agrobacterium tumefaciens or Agrobacterium rhizogenes. Other polynucleotide sequences may be added to, or incorporated within, a border sequence of the present disclosure. Thus, a DNA insert left border, or a DNA insert right border, may be modified so as to possess 5′-and 3′-multiple cloning sites, or additional restriction sites. A DNA insert border sequence may be modified to increase the likelihood that backbone DNA from the accompanying vector is not integrated into the plant genome.

“Commercial production field” or “fruiting field” refers to a field or environment where strawberry plants are grown for fruit production.

“Conditioned” or “conditioning” refers to the process of growing strawberry plug plants such that the plants undergo vernalization. In some embodiments, conditioning occurs in nursery located at high elevations (3000-5000 feet) for a short growing season. In another embodiment, for strawberry production in the northern hemisphere, conditioning occurs in nursery located at northern latitudes with short days and cooler temperatures. In another embodiment, for strawberry production in the southern hemisphere, conditioning occurs in nursery located at southern latitudes with short days and cooler temperatures. In yet another embodiment, the conditioning occurs in a controlled environment with both light exposure and temperature controlled to induce vernalization. The conditioning or vernalization occurs prior to transferring plug plants to the strawberry production field. In some embodiments, the temperature for conditioning is <−2° C., or <−2.2° C., or <−1.5° C., or <0° C., or <5° C., or about 6° C., or about 7° C., or about 8° C., or about 9° C., or about 10° C., or about 11° C., or about 12° C., or about 13° C., or about 14° C., or about 15° C., or about 16° C., or about 17° C., or about 18° C., or about 19° C., or about 20° C., or about 21° C., or about 22° C., or about 23° C., or about 24° C., or about 25° C., or about 26° C., or about 27° C., or about 28° C., or about 29° C., or about 30° C., or between 1° C. and 2° C., or between −2° C. and 5° C., or between −2° C. and 7° C., or between −2° C. and 9° C., or between −2° C. and 10, or between 10-15° C., or between 15-18° C. In some embodiments the day/night temperature is about 18° C./14, or about 30° C./25° C., or about 30° C./26° C. In some embodiments, the length of time for conditioning is 7-35 days, 0-2 weeks, or 3-4 weeks, or 2-10 months, or 3-6 months, or 4-6 months, or 9 months, or longer. In some embodiments, conditioning is done for an Accumulative Chilling Unit (ACU) (° C. hr) (Tanino and Wang (2008)). In some embodiments, the ACU is between 70 and 200° C. hr, or between 100 and 600° C. hr, or between 200 and 1500° C. hr. In some embodiments, the photoperiod is a long day (LD), where LD is about >12 hours of illumination per 24 hour. In some embodiments, the photoperiod about 12, or about 13, or about 14, or about 15 or about 16, or about 17, or about 18, or about 19, or about 20, or about 21, or about 22, or about 23, or 24 hours. In some embodiments, the photoperiod is a short day (SD), where SD is about <12 hours of illumination per 24 hour. In some embodiments, a SD is <12 hours, or about 11 hours, or about 10 hours, or about 9 hours, or about 8 hours, or about 8-12 hours, or about 7 hours, or about 6 hours, or <6 hours. In some embodiments, a photoperiod is a facultative SD, where the illumination is <14 hours. The nursery where conditioning occurs is not limited to a field. The substrate for growing the plug plants may be soil, a field, or an artificial growth medium. The usual customary industry standard for conditioning applies to the process of growing plug plants in nursery fields where the use of a fumigant pesticide, such as methyl bromide, is used. Conditioning is also known as vernalization. In some embodiments, a genetically modified plant of the present application flowers independently of vernalization. In some embodiments, the genetically modified plant flowers independently of temperature and/or photoperiod. As used herein, the term “independently of vernalization” or “independently of temperature and/or photoperiod” refers to the situation that the plant does not experience conditioning, which encompasses the situation where the plant may have been subjected to low temperature briefly, but does not receive enough Accumulative Chilling Unit (e.g., the plant receives an ACU less than 70° C. hr, less than 100° C. hr, or less than 200° C. hr.), or the situation where the plant the plant may have been subjected to photoperiod conditions briefly, but the duration is so short that it does not materially change the flowering time of the plants.

“Cross”, “crossing”, “cross pollination” or “cross-breeding” refer to the process by which the pollen of one flower on one plant is applied (artificially or naturally) to the ovule (stigma) of a flower on another plant.

“Day neutral” refers to a plant that produces flowers regardless of the length of the period of light exposure. A day neutral variety is sometimes referred to as a perpetual flowering variety, or a recurrent variety, or a remontant variety (repeat flowering), or an ever-bearing variety, or a long-day variety.

“Dicotyledon (dicot)” is a flowering plant whose embryo has two seed leaves or cotyledons. Examples of dicots include, but are not limited to, tobacco, tomato, potato, sweet potato, cassaya, legumes including alfalfa and soy bean, carrot, strawberry, lettuce, oak, maple, walnut, rose, mint, squash, daisy, and cactus.

“Earth” means any environment where rooting of a plant takes place, including natural vegetation or synthetic substrate. Earth may refer to the substrate in which the plug plant roots will form a root ball.

“Everbearing” refers to a strawberry variety that produces two or three harvests of strawberry fruit per year, one in the spring and another in the late summer or fall, and under ideal conditions, a third harvest.

“Field” refers to an area of open land where crops are grown or cultivated. The crops are generally planted directly into the soil of the field.

“Fruiting field” or “commercial production field” refers to a field or environment where strawberry plants are grown for fruit production.

“Foreign,” or “exogenous” with respect to a nucleic acid, means that that nucleic acid is derived from non-plant organisms, or derived from a plant that is not the same species as the plant to be transformed, or is derived from a plant that is not interfertile with the plant to be transformed, or does not belong to the species of the target plant. According to the present disclosure, foreign DNA or RNA represents nucleic acids that are naturally occurring in the genetic makeup of fungi, bacteria, viruses, mammals, fish or birds, or plants that is not the same species, but are not naturally occurring in the specific plant that is to be transformed. Thus, a foreign nucleic acid is one that encodes, for instance, a polypeptide that is not naturally produced by the transformed plant.

“Fumigant pesticides” are chemical pesticides traditionally used to treat crop fields to manage soil borne pests. In commercial strawberry production fields, non-limiting examples of the fumigant pesticides used are one of, or a combination of, the following: methyl bromide, 1,3-dichloropropene, trichloronitromethane, chloropicrin, methyl iodide, tetrahydro-3,5-dimethyl-2 H-1,3,5-thiadiazine-2-thione, sodium N-methyl dithiocarbamate, and potassium N-methyl dithiocarbamate.

“Gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

“Genome” refers to the complete DNA component of an organism. In plants, a genome may be a nuclear genome, a chloroplast genome, or a mitochondrial genome.

“Genetically modified” refers to a non-natural change in a genome of an organism. In one embodiment, a modification is induced by a mutagen. In some embodiments, a modification to a genome is by genome editing. In some embodiments, a modification to a genome is with transient transformation. In some embodiments, a modification to a genome is by stable transformation. In some embodiments, an expression cassette is integrated into the genome of the organism.

“Growing” means the process in which a plant goes from seed to the time death occurs, including any such time in between where dormancy is taking place.

“High elevation” refers to an elevation within a range of about 3000 to 6000 feet above sea level, ideally around 5000 feet above sea level. As used here, high elevation applies to the geographical elevation where strawberry nursery fields are located.

“Homologous” or “homologue” or “ortholog” is known in the art and refers to related sequences that share a common ancestor or family member and are determined based on the degree of sequence identity. The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure, homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. The degree of sequence identity may vary, but in some embodiments, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters. Where a particular sequence is said to have a specific percent identity to a reference sequence of a defined length, the percent identity is relative to the reference sequence. Thus, a sequence that is 50% identical to a reference sequence that is 100 amino acids (or 100 nucleotides long) can be a 50 amino acid polypeptide or a 50 nucleotide sequence that is completely identical to a 50 amino acid long portion of the reference polypeptide or a 50 nucleotides long portion of the reference nucleotide sequence. It might also be a 100 amino acid long polypeptide, or a 100 nucleotide sequence, which is 50% identical to the reference polypeptide or the reference nucleotide sequence over its entire length. Of course, other sequences unspecified may also meet the same criteria.

“Integrate” refers to the insertion of a nucleic acid sequence from a selected plant species, or from a plant that is from the same species as the selected plant, or from a plant that is sexually compatible with the selected plant species, into the genome of a cell of a selected plant species. “Integration” refers to the incorporation of only native genetic elements into a plant cell genome. In order to integrate a native genetic element, such as by homologous recombination, the present disclosure may “use” non-native DNA as a step in such a process. Thus, the present disclosure distinguishes between the “use of” a particular DNA molecule and the “integration” of a particular DNA molecule into a plant cell genome.

“Introduction” refers to the insertion of a nucleic acid sequence into a cell, by methods including infection, transfection, transformation or transduction.

“Northern latitude” is a geographical latitude in the earth's northern hemisphere where strawberry nursery fields are located and where the strawberry nursery plants undergo vernalization. As used herein, a “southern latitude” is a geographical latitude in the earth's southern hemisphere where strawberry nursery fields are located and where the strawberry nursery plants undergo vernalization.

“June-Bearing” refers to a strawberry variety that produces fruit around the month of June. The June-bearing strawberry varieties can be divided into ‘early season’, ‘early midseason’, ‘midseason’, ‘late midseason’, and ‘late season’ referring to the relative timing of when fruiting begins. For example, relative to the early season varieties, fruiting begins about 5 days later for the early midseason variety; fruiting begins about 8 days later for the midseason varieties; fruiting begins about 10 days later for the late midseason varieties; and fruiting begins about 14 days later for the late season varieties. June-bearing varieties may also be referred to as a short-day variety or a seasonal flowering variety.

“Locus”. A locus confers one or more traits such as, for example, male sterility, herbicide tolerance, insect resistance, disease resistance, waxy starch, modified fatty acid metabolism, modified phytic acid metabolism, modified carbohydrate metabolism, and modified protein metabolism. The trait may be, for example, conferred by a naturally occurring gene introduced into the genome of the variety by backcrossing, a natural or induced mutation, or a transgene introduced through genetic transformation techniques. A locus may comprise one or more alleles integrated at a single chromosomal location.

“Long day” is a 24 hour period (a day) with more than 12 hours of light.

“Low elevation” refers to an elevation of less than sea level to about 3000 feet above sea level, ideally below about 600 feet above sea level. As used here, low elevation applies to the geographical elevation where strawberry production fields are located.

“Methyl bromide” is a fumigant pesticide commonly used as a pesticide during strawberry plug plant development. Methyl bromide has a chemical formula of CH₃BR, and a CAS number 74-83-9.

“Non-natural mutant” refers to mutants created by any means that involves at least some minimum human intervention. For example, non-natural mutants can be created by direct human intervention, or indirect intervention. Non-natural mutants can be created by using at least some human-made compositions, tools, or procedures not naturally existing. Non-natural mutants can be created by any suitable mutagenesis method known to one skilled in the art, and any suitable method that can be used to alter the function of one or more genes in a given plant. The alteration of the gene function can be at different levels, including but not limited to, gene expression level, RNA activity level, or protein activity level. As used herein, the term “RNA activity level” refers to mRNA abundance, synthesis rate, and/or stability. As used herein, the term “protein activity level” refers to protein abundance, synthesis rate, stability, enzymatic activity, phosphorylation rate.

“Nursery” or “nursery field” refers to a location where plants are propagated and grown to a usable size in a production field. In some embodiments, a nursery is a greenhouse, screenhouse, or other controlled environment facility. In some embodiment, a nursery is a field. In some embodiments, plants in a nursery field undergo vernalization. In other embodiments, plants in a nursery field do not undergo vernalization. In some embodiments, a nursery field is at a high elevation. In some embodiments, a nursery field is at a low elevation. In some embodiments, a nursery field in the Northern latitude is at geographical latitude in the earth's northern hemisphere where plants undergo vernalization. In some embodiments, a nursery field in the Southern latitude is at geographical latitude in the earth's southern hemisphere where plants undergo vernalization. In some environments, strawberry plug plants are produced in a nursery or nursery field.

“Offspring” refers to any plant resulting as progeny from a vegetative or sexual reproduction from one or more parent plants or descendants thereof. For instance an offspring plant may be obtained by cloning or selfing of a parent plant or by crossing two parental plants and include selfings as well as the F1 or F2 or still further generations. An F1 is a first-generation offspring produced from parents at least one of which is used for the first time as donor of a trait, while offspring of second generation (F2) or subsequent generations (F3, F4) are specimens produced from selfings of F1's, F2's. An F1 may thus be (and usually is) a hybrid resulting from a cross between two true breeding parents (true-breeding is homozygous for a trait), while an F2 may be (and usually is) an offspring resulting from self-pollination of said F1 hybrids.

“Operably linked” refers to combining two or more molecules in such a fashion that in combination they function properly in a plant cell. For instance, a promoter is operably linked to a structural gene when the promoter controls transcription of the structural gene.

“Overexpress” or “overexpression” refers to the process of inducing expression of a gene/protein beyond its normal endogenous activity. For instance, overexpression of a gene as described herein may be used to increase flowering. Overexpress or overexpression refers to any means that can increase the activity of a target gene when compared to the activity of a check gene (for example, a wild type allele in the same plant species). The increase can be at gene expression level, RNA activity level, and/or protein activity level, including but not limited to, increased gene copy number, increased gene amplification, increased mRNA abundance, synthesis rate, and/or stability, increased protein synthesis, protein abundance, stability, enzymatic activity, or phosphorylation. In some embodiments, overexpression happens when a copy of the target gene or its homolog is introduced in the plant. In some embodiments, overexpression happens when extra chromosomes or portions of chromosomes bearing the target gene is introduced in the plant. In some embodiments, overexpression happens when an enhancer for expression is used. The definition also encompasses ectopic expression and any means that lead to upregulation of the gene activity.

“Photoperiod” refers to the length of time in a 24 hour cycle that a plant receives illumination. In some embodiments, a photoperiod is a short day with less than 12 hours of illumination per 24 hour period. In some embodiments, a photoperiod is a long day with more than 12 hours of illumination per 24 hour period.

Plant: the disclosure provides compositions and methods to produce plants having modified phenotypes when compared to a wild-type control plant. As used herein, the term “plant” refers to a plant in the Rosaceae family, such as plants in the genus of Rosa or Fragaria, unless specified.

“Plant part” refers to any part of a plant including but not limited to the embryo, shoot, root, stem, seed, stipule, leaf, petiole, petal, calyx, sepal, flower, ovule, bract, branch, internode, pubescence, tiller, rhizome, frond, blade, ovule, pollen, stamen, runner, stolon, achene.

“Plug plants” are young plants grown with the intent of being replanted in a secondary location. Plug plants have a characteristic root ball that improves the chances for survival after transplanting, and increases the growth rate after transplanting into the fruit production field. Plug plants are also referred to as a daughter plants.

Polynucleotide: the disclosure provides isolated, chimeric, recombinant or synthetic polynucleotide sequences. As used herein, the terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

A “plant promoter” is a promoter capable of initiating transcription in plant cells whether or not its origin is a plant cell, for example, it is well known that Agrobacterium promoters are functional in plant cells. Thus, plant promoters include promoter DNA obtained from plants, plant viruses and bacteria such as Agrobacterium and Bradyrhizobium bacteria. A plant promoter can be a constitutive promoter or a non-constitutive promoter.

A “constitutive promoter” is a promoter which is active under most conditions and/or during most development stages. There are several advantages to using constitutive promoters in expression vectors used in plant biotechnology, such as: high level of production of proteins used to select transgenic cells or plants; high level of expression of reporter proteins or scorable markers, allowing easy detection and quantification; high level of production of a transcription factor that is part of a regulatory transcription system; production of compounds that requires ubiquitous activity in the plant; and production of compounds that are required during all stages of plant development. Non-limiting exemplary constitutive promoters include: CaMV 35S promoter, opine promoter, ubiquitin promoter, and alcohol dehydrogenase promoter.

A “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues, such as stems, leaves, roots, or seeds.

An “inducible” or “repressible” promoter is a promoter which is under chemical or environmental factors control. Examples of environmental conditions that may affect transcription by inducible promoters include anaerobic conditions, or certain chemicals, or the presence of light.

A “tissue specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related plant species to achieve efficient and reliable expression of transgenes in particular tissues. This is one of the main reasons for the large amount of tissue-specific promoters isolated from particular plants and tissues found in both scientific and patent literature.

“Synthetic promoter” refers to a promoter that is not naturally found in nature. The nucleotide sequence is artificial or synthetic. A synthetic promoter may be a constitutive promoter, it may be a non-constitutive promoter, it may an inducible promoter, or it may be a tissue specific promoter. Exemplary synthetic promoters useful for transgene expression are disclosed in U.S. Pat. No. 9,670,497, which is herein incorporated by reference in its entirety.

Recombinant: the disclosure also provides chimeric or recombinant molecules for altering gene function in a plant. As used herein, the term “chimeric” or “recombinant” when describing a nucleic acid sequence or a protein sequence refers to a nucleic acid or a protein sequence that links at least two heterologous polynucleotides or two heterologous polypeptides into a single macromolecule, or that re-arranges one or more elements of at least one natural nucleic acid or protein sequence. For example, the term “recombinant” can refer to an artificial combination of two otherwise separated segments of sequence, for example, by chemical synthesis or by the manipulation of isolated segments of nucleic acids by genetic engineering techniques.

“Repress” or “repression” refers to any mean that can reduce the activity of a target gene when compared to the activity of a check gene (for example, a wild type allele in the same plant species). The reduction can be at gene expression level, RNA activity level, and/or protein activity level, including but not limited to, reduced gene copy number, reduced gene amplification, reduced mRNA abundance, synthesis rate, and/or stability, reduced protein synthesis, protein abundance, stability, enzymatic activity, or phosphorylation. In some embodiments, the repression happens when a DNA-binding repressor blocks the attachment of RNA polymerase to the promoter, thus preventing transcription of the genes into messenger RNA. In some embodiments, the repression happens when one or more mutations are introduced into the promoter/coding/intron or terminator region of the target gene. In some embodiments, the repression happens when interference RNA is introduced into the plant to inhibit the target gene. The definition also encompasses varies degrees of modified gene activity, such as modified gene activity achieved by gene silencing, loss-of-function mutant, knock-out, knock-down, leaky mutation. The degree to which the function of a target gene is lost can vary. For example, the target gene can completely lose its function (for example, a null mutation), or partially maintain its function, but not at the level of a wild-type check allele (for example, a leaky mutation). In the present disclosure, for instance, repression of a TFL gene, such as TFL1, using known techniques results in reduced TFL activity.

“Gene activity” refers to gene expression level, RNA activity level, or protein activity level. As used herein, the term “RNA activity level refers to mRNA abundance, synthesis rate, and/or stability. As used herein, the term “protein activity level” refers to protein abundance, synthesis rate, stability, enzymatic activity, phosphorylation rate.

“Root ball” is a spherically shaped mass of a plant's root system. For the present disclosure strawberry plug plants are grown in such a way to produce a desirable root ball for the purpose of increasing the survival and health of the plant after replanting.

“Season” refers to the time of the year in which a plant is actively growing in size, undergoing phenotypic changes, and is therefore not dormant.

“Sequence identity” or “identity” in the context of two nucleic acid or polypeptide sequences includes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4:11-17 (1988).

“Short day” is a 24 hour period (a day) with less than 12 hours of light.

“Soil” refers to the layer of earth in a field where crops are grown. In some embodiments, the crops are directly planted in the soil. In other embodiments, the soil is placed in a container, for example, a pot or elevated growing vessel, and plants are grown in the soil in the pot or elevated growing vessel. In some embodiments, the soil may be ‘natural’, meaning taken directly from the field. In other embodiments, the soil may be may be amended with compost, peat, sand, clay, organic matter, in-organic matter, fertilizer, or other composition to aid in plant growth. In some embodiments, plants of the present disclosure are grown in a hydroponic condition.

“Strawberries” are plants whose fruits are juicy, edible, low growing, and of genus Fragaria. According to this present disclosure, a strawberry is the desired product to be harvested from a plug plant grown at low elevation.

“Stringent hybridization” or “stringency” or “stringent hybridization conditions” refer to hybridization conditions that affect the stability of hybrids, for example, temperature, salt concentration, pH, and formamide concentration. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (for example, at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na+ ion, typically about 0.01 to 1.0 M Na⁺ ion concentration (or other salts) at pH 7.0 to 8.3, and the temperature is at least about 30° C. for short probes or primers (for example, 10 to 50 nucleotides), and at least about 60° C. for long probes or primers (for example, greater than 50 nucleotides). Stringent conditions may also be achieved with the addition of destabilizing agents such as formamide. Generally, stringent conditions are selected to be about 5° C. lower than the thermal melting point (Tm) for the specific sequence and its complement at a defined ionic strength and pH. However, severely stringent conditions can utilize a hybridization and/or wash at 1° C., 2° C., 3° C., or 4° C. lower than the thermal melting point (Tm); moderately stringent conditions can utilize a hybridization and/or wash at 6° C., 7° C., 8° C., 9° C., or 10° C. lower than the thermal melting point (Tm); low stringency conditions can utilize a hybridization and/or wash at 11° C., 12° C., 13° C., 14° C., 15° C., or 20° C. lower than the thermal melting point (Tm). Exemplary low stringent conditions or “conditions of reduced stringency” include hybridization with a buffer solution of 30% formamide, 1 M NaCl, 1% SDS at 37° C., and a wash in 2× Standard Sodium Citrate (SSC) at 40° C. Exemplary high stringency conditions include hybridization in 50% formamide, 1M NaCl, 1% SDS at 37° C., and a wash in 0.1×SSC at 60° C. Hybridization procedures are well known in the art and are described by Ausubel et al., 1998 and Sambrook et al., 2001. In some embodiments, stringent conditions are hybridization in 0.25 M Na₂HPO4 buffer (pH 7.2) containing 1 mM Na₂EDTA, 0.5-20% sodium dodecyl sulfate at 45° C., such as 0.5%, 1%, 2%, 3%, 4%, 5%, 6%, 7%, 8%, 9%, 10%, 11%, 12%, 13%, 14%, 15%, 16%, 17%, 18%, 19%, or 20%, followed by a wash in 5×SSC, containing 0.1% (w/v) sodium dodecyl sulfate, at 55° C. to 65° C. In the present disclosure, homologs of TFL genes that hybridize with a strawberry gene under stringent hybridization conditions are contemplated.

“Substrate” refers to a composition in which plants are grown. In some embodiments, substrate is soil. In another embodiment, the substrate is a field. In yet another embodiment, a substrate is soil amended with other components, such as compost, peat, sand, clay, organic matter, in-organic matter, fertilizer, or other composition to aid in plant growth. In some embodiments, a substrate is natural, meaning as found in nature. In other embodiments, a substrate is a composition not found in nature. In some embodiments, a substrate is a mixture of both natural and non-natural compositions. In some embodiments, the substrate is a hydroponic solution.

The TERMINAL FLOWER gene, “TFL” encodes a transcription factor that functions as a suppressor of flowering. In strawberry, expression of endogenous TFL is regulated by temperature and photoperiod. For example, in short-day varieties, conditioning the strawberry plants at low temperature and with a short day photoperiod reduces expression of TFL. Reduction of TFL releases the suppression of flowering, and flower induction proceeds. The specific parameters of temperature and photoperiod that regulate TFL vary by strawberry variety and have been reviewed by Heide and Sonsteby (2014). The present inventive method downregulates activity of endogenous TFL. Therefore, there is less of the TFL transcription factor available to suppress flowering. Accordingly, these genetically modified strawberries, with downregulated TFL, flower independently of temperature and photoperiod.

As used herein, TFL encompasses functional variants and homologs of TFL genes. There are three homologs of TFL: TFL1 (as represented by SEQ ID NOs: 1 and 4 (genomic DNA), SEQ ID Nos: 2, 5, and 7 (cDNA), and SEQ ID NOs: 3, 6, and 8 (PRT)), TFL2 (as represented by SEQ ID NO: 9 (DNA) and SEQ ID NO:10 (PRT)), and TFL3 (as represented by SEQ ID NO: 11 (DNA) and SEQ ID NO: 12 (PRT)), but other homologs may be used according to the methods disclosed herein. Thus, the term ‘TFL gene’ also refers to sequences having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity to SEQ ID NOs. 1, 4, 2, 5, 7, 9, or 11, any sequence encoding SEQ ID NOs. 3, 6, 8, 10, and 12, and sequences that can hybrid to any of the sequences mentioned above under stringent hybridization conditions, wherein the sequences encode a functional TFL protein.

The Flowering Locus T gene, “FT”, encodes a protein that functions as an activator of flowering. In strawberry, expression of endogenous FT is regulated by temperature and photoperiod. For example, in short-day varieties, conditioning the strawberry plants at low temperature and with a short day photoperiod increases expression of FT. Induction of FT promotes the flowering. The specific parameters of temperature and photoperiod have been reviewed by Nakano et al. (J Plant Physiol. 2015 Apr. 1; 177:60-6). The present inventive method upregulates the activity of FT. Accordingly, these genetically modified strawberries, with upregulated FT, flower independently of temperature and photoperiod.

As used herein, FT encompasses functional variants and homologs of FT genes. Thus, the term ‘FT gene’ also refers to sequences having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identity to SEQ ID NOs. 22, SEQ ID NO: 24, or SEQ ID NO: 25, or a gene encoding SEQ ID NO: 23 or SEQ ID NO: 26, and sequences that can hybrid to any of the sequences mentioned above under stringent hybridization conditions, wherein the sequences encode a functional FT protein.

“Tissue culture” refers to a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, leaves, stems, roots, root tips, anthers, pistils, meristematic cells, axillary buds, ovaries, seed coat, endosperm, hypocotyls, cotyledons and the like. Means for preparing and maintaining plant tissue culture are well known in the art. By way of example, a tissue culture comprising organs has been used to produce regenerated plants. U.S. Pat. Nos. 5,750,870, 5,959,185, 5,973,234, and 5,977,445 describe certain techniques, the disclosures of which are incorporated herein by reference.

“Vernalization” is the process of promoting flowering by exposing the strawberry plants to prolonged chilling and/or controlled photoperiods. The process of vernalization may also be referred to as ‘conditioning’ in strawberry production.

“Yield” refers to the weight of fruit harvested. Yield can be measured by the number of fruit, weight of fruit, or the number and weight of fruit harvested per plant, or per acre of plants, within a given period of time, such as a season or a year.

“500 feet” means an elevation of 500 feet above sea level.

In some embodiments, plants with modified genes of the present disclosure are strawberry plants. As used herein, the term strawberry encompasses plant species in the Fragaria genus. The genetics of strawberry plants are uniquely diverse in terms of ploidy. Strawberry plant species can be diploid, tetraploid, pentaploid, hexaploid, heptaploid, octaploid, or decaploid (which have 2, 4, 5, 6, 7, 8, or 10 sets of chromosomes, respectively). Some species of Fragaria have uncategorized ploidy. Fragaria, known as strawberry is a genus of flowering plants in the rose family, Rosaceae.

Fragaria species include but are not limited to, 1) diploid: F. bucharica, F. chinensis, F. daltoniana, F. gracilis, F. hayatai, F. iinumae, F. nilgerrensis, F. nipponica, F. nubicola, F. pentaphylla, F. rubicola, F. vesca, F. viridis, F. vezoensis, and F.×bifera; 2) tetraploid: F. corymbosa, F. moupinensis, F. orientalis, and F. tibetica; 3) pentaploid: F.×bringhurstii; 4) hexaploid: F. moschata; 5) hexaploid: F. moschata; 6) heptaploid: F.×comarum; 7) octaploid: F.×ananassa, F. chiloensis, F. chiloensis subsp. chiloensis forma chiloensis, F. chiloensis subsp. chiloensis forma patagonica, F. chiloensis subsp. Lucida, F. chiloensis subsp. Pacifica, Fragaria chiloensis subsp. Sandwicensis, F. iturupensis, F. ovalis, and F. virginiana; 8) decaploid: C. frutescens, C. chinense, and C. pendulum. More Fragaria species are described in Liston et al. (Fragaria: A genus with deep historical roots and ripe for evolutionary and ecological insights, American Journal of Botany (2014) 101:1686-1699) and Kole (Wild Crop relatives: Genomic and Breeding Resources: Temperate Fruits, Chapter 2 Fragaria, 2011).

The growing of strawberry plug plants with reduced activity of endogenous TERMINAL FLOWER (TFL) gene(s) (for example, reduced expression), and/or induced activity of FT gene, allows cultivation where flowering is independent of temperature and photoperiod, increases yield, decreases delay-time in plug plant production, reduces or eliminates the need for the use of fumigant pesticides, and does not produce undesirable phenotypes or harmful side-effects. Thus, the present disclosure encompasses plants, and methods for making such plants with reduced expression of endogenous TERMINAL FLOWER (TFL) gene(s).

In plants of the present disclosure, the flowering time signaling pathway is modified, compared to a control plant without the modification. In some embodiments, one or more genes that are involved in flowering time signaling pathway are modified.

Flowering in plants is determined by the timing of the transition from vegetative to reproductive stage. Endogenous signal transduction cascade initiated or altered by environmental factors causes this phase change during the life cycle of a plant. A set of genes involved in signal cascades finely regulate the transition to flowering. The genetic and molecular interaction of key players can control flowering time and floral organ identity. TFL represses floral meristem identity genes LFY (Leafy) and AP1 (Apetalal), while FT promotes flowering by activating LFY and AP1 in response to long days. Repression of TFL activity or mutation in TFL demonstrates upregulation of its target genes, LFY and AP1, resulting in early flowering. Also, ectopic expression of LFY or AP1 leads to forming a terminal flower resembling plants in which TFL is mutated. Suppression of TFL gene also causes increased expression of FT. Further study on the overexpression of FT illustrates early flowering, which is similar to tfl mutant phenotype. Thus, the opposite effects of TFL1 and FT on flowering illustrate that the balance between FT and TFL1 activity plays a role in governing the integration of floral inductive signals and the decision of flowering time. For an additional summary of the flowering time signaling pathway, see Bradley et al., (1997) Science 275:80-83; Ruiz-Garcia et al. (1997) Plant Cell 9:1921-1934; Corbesier and Coupland, (2005) Plant Cell and Environment 28:54-66; Mandel and Yanofsky, (1995) Nature 377:522-524; Weigel and Nilsson, (1995) Nature 377:495-500; Kardailsky et al., (1999) Science 286:1962-1965; Iwata et al., (2012) The Plant J. 69:116-125; Koskela et al., (2012) Plant Physiol. 159:1043-1054; and Nakano et al., (2015) J. Plant Physiol.177:60-66. In some embodiments of the present disclosure, the modified genes are TFL gene, upstream genes that directly or indirectly regulate TFL activity, or downstream genes that are regulated by TFL gene. These genes constitute the TFL gene signal transduction cascade. In some embodiments, as a result of the modification, the activity of TFL in the plant is repressed, and/or a gene that is negatively regulated by TFL is activated, such as LFY or AP1, when compared to a control plant without the modification. In some embodiments, the TFL gene has the sequence of SEQ ID NO. 1, 2, 4, 5, 7, 9, or 11, functional variants or homologs thereof, or sequences encoding a TFL protein having the sequence of SEQ ID NO: 3, 6, 8, 10, or 12. In some embodiments, the TFL gene shares at least about 90%, about 91%, about 92%, about 93%, about 94%, about 95%, about 96%, about 97%, about 98%, about 99%, or more identity to any of SEQ ID NOs. 1, 2, 4, 5, 7, 9, and 11. In some embodiments, the TFL gene encodes a functional TFL protein having at least about 90%, about 91%, about 92%, about 93%, about 94%, about 95%, about 96%, about 97%, about 98%, about 99%, or more identity to any of SEQ ID NOs. 3, 6, 8, 10, and 12.

Alternatively, allelic diversity in genes in the flowering time signaling pathway can be used to achieve the same effect. In some embodiments, genetic variations in the promoter or coding or intron or terminator region of a flowering time signaling pathway gene can be used to identify alleles that have enhanced or repressed activity when compared to a corresponding check allele. For example, a TFL allele found in a plant species that has reduced activity when compared to the TFL allele in a control plant can be utilized to produce a plant with repressed TFL activity, by integrating the TFL allele having reduced activity into a desired plant produced by such incorporations of gene alleles via human intervention are within the definition of “plants having modified flowering time signaling pathway” of the present disclosure.

Repression of TFL1 signal transduction cascade and/or upregulation of FT signal transduction cascade in strawberry plants of the present disclosure produce high-yielding plug plants that flower independently of temperature and photoperiod (for example, they do not require conditioning). Pest-free nursery stock plants/plug plants can be produced without the use of harmful fumigants, without conditioning, and are high yielding. Repression of endogenous TFL activity can be achieved by any suitable methods known in the field, including but not limited to, RNAi and genome editing, by using markers for coding or regulatory regions encompassing promoters, introns, terminators, 5′ and 3′ UTR regions, or any agent that down regulates TFL gene signal transduction cascade activity, such as gibberellins.

Methods for modify gene activity that can be utilized in the present disclosure include, but are not limited to, mutagenesis (for example, chemical mutagenesis, radiation mutagenesis, transposon mutagenesis, insertional mutagenesis, signature tagged mutagenesis, site-directed mutagenesis, and natural mutagenesis), knock-outs/knock-ins, antisense, RNA interference, and any other suitable methods known to a skilled artisan, such as Zinc finger nuclease (ZFN) technology (ZFN-1, ZFN-2 and ZFN-3, see U.S. Pat. No. 9,145,565, incorporated by reference in its entirety), Oligonucleotide directed mutagenesis (ODM), Cisgenesis and intragenesis, RNA-dependent DNA methylation (RdDM), Grafting (on GM rootstock), Reverse breeding, Agro-infiltration (agro-infiltration “sensu stricto”, agro-inoculation, floral dip), Transcription Activator-Like Effector Nucleases (TALEN5, see U.S. Pat. Nos. 8,586,363 and 9,181,535, incorporated by reference in their entireties), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference), engineered meganuclease re-engineered homing endonucleases, DNA guided genome editing (Gao et al., Nature Biotechnology (2016), doi: 10.1038/nbt.3547, incorporated by reference in its entirety), and Synthetic genomics. For more information of gene modification in plants, such as agents, protocols, see Acquaah et al. (Principles of plant genetics and breeding, Wiley-Blackwell, 2007, ISBN 1405136464, 9781405136464, which is herein incorporated by reference in its entity).

In some embodiments, the gene activity is modified by introducing a mutation into the plants. Methods of introducing a mutation into an endogenous gene or replacing an endogenous gene or a portion thereof with a mutant gene are well known in the art. In certain embodiments, a variety of DNA nucleases may be utilized to introduce mutations into an endogenous gene. In certain embodiments, the DNA nuclease is deficient in its nuclease activity. In certain embodiments, the enzyme is a Zinc-finger nuclease. In further embodiments, the Zinc-finger nuclease is ZF-FokI or ZF-Tn3. In certain embodiments, the enzyme is a transcription activator-like effector nuclease (TALEN). In further embodiments, the TALEN is TAL-FokI. In certain embodiments, the enzyme is a homing endonuclease. In further embodiments, the homing endonuclease is LAGLIDADG, GIY-YIG, His-Cys, H-N-H, PD-(D/E)xK, or Vsr-like. In certain embodiments, the enzyme is an RNA-guided nuclease such as a Clustered Regularly Interspaced Short Palindromic Repeats (CRISPR) enzyme. In further embodiments, the CRISPR enzyme is a type II CRISPR enzyme. In further embodiments, the type II CRISPR enzyme is Cas9. In certain embodiments, the CRISPR enzyme is deficient in its nuclease activity. In certain embodiments, various DNA integrases may be utilized to introduce mutations into an endogenous gene or replace an endogenous gene with a mutant gene. In certain embodiments, the DNA integrase is λ-int or φC31. In certain embodiments, a DNA recombinase may be utilized to introduce mutations into an endogenous gene or replace an endogenous gene with a mutant gene. In certain embodiments, the DNA recombinase is Cre, Flp, or RMCE. In other embodiments, the Cas9 peptide can include one or more of the mutations described in the literature, including but not limited to the functional mutations described in: Fonfara et al. (2014) Nucleic Acids Res. 42(4):2577-90; Nishimasu H. et al. (2014) Cell. 156(5):935-49; Jinek M. et al. (2012) Science 337:816-21; and Jinek M. et al. (2014) Science 343(6176); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference.

In some embodiments, the activity of one or more genes in the flowering time signaling pathway is disrupted by using an inhibitory nucleotide sequence, such as nucleotide sequences for RNA interference, antisense oligonucleotides, microRNA, and/or steric-blocking oligonucleotides (See Kole et al., (2012) Drug Discovery 11:125-140; Ossowski et al. (2008) The Plant Journal, 53(4):674-690; Wang et al. (2002) Current Opinion in Plant Biology, 5(2):146-150; Vaucheret et al. (2001) Journal of Cell Science 114:3083-3091; Stam et al. (1997) Annals of Botany 79(1):3-12; Schwab et al. (2006) The Plant Cell 18(5):1121-1133; C. David Allis et al., Epigenetics, CSHL Press (2007) ISBN 10: 0879697245, ISBN 13: 978087969724; Sohail et al., Gene silencing by RNA interference: technology and application, CRC Press (2005) ISBN 0849321417, 9780849321412; Engelke et al., RAN Interference, Academic Press (2005) ISBN 0121827976, 9780121827977; and Doran et al., RNA Interference: Methods for Plants and Animals, CABI (2009) ISBN 1845934105, 9781845934101, each of which is incorporated herein by reference in its entirety for all purposes). In some embodiments, one or more genes in the flowering time signaling pathway are disrupted by RNA interference (RNAi). RNAi is the process of sequence-specific, post-transcriptional gene silencing or transcriptional gene silencing in animals and plants, initiated by double-stranded RNA (dsRNA) that is homologous in sequence to the silenced gene. The preferred RNA effector molecules useful in this disclosure must be sufficiently distinct in sequence from any host polynucleotide sequences for which function is intended to be undisturbed after any of the methods of this disclosure are performed. Computer algorithms may be used to define the essential lack of homology between the RNA molecule polynucleotide sequence and host, essential, normal sequences.

In some embodiments, one or more genes in the flowering time signaling pathway are disrupted by double-strand RNA. The term “dsRNA” or “dsRNA molecule” or “double-strand RNA effector molecule” refers to an at least partially double-strand ribonucleic acid molecule containing a region of at least about 19 or more nucleotides that are in a double-strand conformation. The double-stranded RNA effector molecule may be a duplex double-stranded RNA formed from two separate RNA strands or it may be a single RNA strand with regions of self-complementarity capable of assuming an at least partially double-stranded hairpin conformation (for example, a hairpin dsRNA or stem-loop dsRNA). In various embodiments, the dsRNA consists entirely of ribonucleotides or consists of a mixture of ribonucleotides and deoxynucleotides, such as RNA/DNA hybrids. The dsRNA may be a single molecule with regions of self-complementarity such that nucleotides in one segment of the molecule base pair with nucleotides in another segment of the molecule. In one aspect, the regions of self-complementarity are linked by a region of at least about 3-4 nucleotides, or about 5, 6, 7, 9 to 15 nucleotides or more, which lacks complementarity to another part of the molecule and thus remains single-stranded (for example, the “loop region”). Such a molecule will assume a partially double-stranded stem-loop structure, optionally, with short single stranded 5′ and/or 3′ ends. In one aspect the regions of self-complementarity of the hairpin dsRNA or the double-stranded region of a duplex dsRNA will comprise an Effector Sequence and an Effector Complement (for example, linked by a single-stranded loop region in a hairpin dsRNA). The Effector Sequence or Effector Strand is that strand of the double-stranded region or duplex which is incorporated in or associates with RISC. In one aspect the double-stranded RNA effector molecule will comprise an at least 19 contiguous nucleotide effector sequence, preferably 19 to 29, 19 to 27, or 19 to 21 or more nucleotides, which is a reverse complement to the RNA of the target gene, or an opposite strand replication intermediate. In some embodiments, the dsRNA effector molecule of the disclosure is a “hairpin dsRNA”, a “dsRNA hairpin”, “short-hairpin RNA” or “shRNA”, for example, an RNA molecule of less than approximately 400 to 500 nucleotides (nt), or less than 100 to 200 nt, in which at least one stretch of at least 15 to 100 nucleotides (for example, 17 to 50 nt, 19 to 29 nt) is based paired with a complementary sequence located on the same RNA molecule (single RNA strand), and where said sequence and complementary sequence are separated by an unpaired region of at least about 4 to 7 nucleotides (or about 9 to about 15 nt, about 15 to about 100 nt, about 100 to about 1000 nt) which forms a single-stranded loop above the stem structure created by the two regions of base complementarity. The shRNA molecules comprise at least one stem-loop structure comprising a double-stranded stem region of about 17 to about 500 bp; about 17 to about 50 bp; about 40 to about 100 bp; about 18 to about 40 bp; or from about 19 to about 29 bp; homologous and complementary to a target sequence to be inhibited; and an unpaired loop region of at least about 4 to 7 nucleotides, or about 9 to about 15 nucleotides, about 15 to about 100 nt, about 250-500 bp, about 100 to about 1000 nt, which forms a single-stranded loop above the stem structure created by the two regions of base complementarity. It will be recognized, however, that it is not strictly necessary to include a “loop region” or “loop sequence” because an RNA molecule comprising a sequence followed immediately by its reverse complement will tend to assume a stem-loop conformation even when not separated by an irrelevant “stuffer” sequence. In yet another embodiment, the RNA interference is through use of an RNA “trigger”, such as described in U.S. Patent Application Publications US20140215656; US20130067618A1; US20130288895A1; US20130254940A1; US20130097726; US20130326731A1, all of which are incorporated herein in their entirety.

In some embodiments, the RNAi constructs of the present disclosure comprise one or more inverted repeats. The inverted repeats can be transcribed into interference RNA molecules in the plants. In some embodiments, the transcribed interference RNA molecules can target the promoter region, the coding region, the intron, the 5′ UTR region, and/or the 3′ UTR region of a gene in the TFL signal transduction cascade, such as a TFL gene (TFL1, TFL2, and/or TFL3), or a gene in the upstream that induces TFL activity, such as CO.

In some embodiments, the inverted repeats comprise a sense strand and an anti-sense strand. In some embodiments, the sense stand and the anti-sense stand are perfectly complementary to each other. In some embodiments, the sense stand and the anti-sense stand are not perfectly complementary to each other for the full length, but are at least complementary partially. In some embodiments, the sense stand shares about 70%, about 80%, about 90%, about 95%, about 99% or more homology to the targeted gene.

In some embodiments, plants in which a flowering time signaling pathway gene is modified have one or more agriculturally important traits. As used herein, “agronomically important traits” include any phenotype in a plant or plant part that is useful or advantageous for human use. Examples of agronomically important traits include but are not limited to those that result in increased biomass production, increased food production, improved food quality, increased fruit production. Additional examples of agronomically important traits includes pest resistance, vigor, development time (time to harvest), enhanced nutrient content, novel growth patterns, flavors or colors, salt, heat, drought and cold tolerance, disease resistance, fruit size, fruit weight, fruit color, fruit nutrients, fruit taste, and the like. Non-limited examples of disease resistance include, resistant to Raspberry ringspot virus (RpRSV), Strawberry crinkle virus (SCV), Strawberry feather leaf virus, Strawberry latent “C” virus (SLCV), Strawberry latent ringspot virus (SLRSV), Strawberry leaf roll virus, Strawberry mild yellow edge virus (SMYEV), Strawberry mottle virus (SMV), Strawberry pallidosis virus, Strawberry vein banding virus (SVBV), Tobacco necrosis virus (TNV), Tobacco ringspot virus (TRSV), Tobacco streak virus (TSV), Strawberry necrotic shock virus (SNSV), Tomato black ring virus (TBRV), Tomato bushy stunt virus (TBSV), Tomato ringspot virus (ToRSV), and Xanthamonas fragariae (angular leafspot). Additional preferred traits are described in Yue et al. (An Evaluation of U.S. Strawberry Producers Trait Prioritization: Evidence from Audience Surveys, HortScience 49(2) 188-193 (2014)).

Also provided are expression vectors that can be used to produce the plants of the present disclosure. The backbone of the expression vectors can be any expression vectors suitable for producing transgenic plant. In some embodiments, the expression vector is suitable for expressing transgene in strawberry plants.

In some embodiments, the expression vector is an Agrobacterium binary vector (see, Karimi et al., (2007) Plant Physiol 145: 1183-1191; Komari et al., (2006) Methods Mol Biol 343: 15-42; Bevan M W (1984) Nucleic Acids Res 12: 1811-1821; Becker (1992), Plant Mol Biol 20: 1195-1197; Datla et al., (1992), Gene 122: 383-384; Hajdukiewicz (1994) Plant Mot Blot 25:989-994; Xiang (1999), Plant Mot Blot 40: 711-717; Chen et al., (2003) Mol Breed 11: 287-293; Weigel et al., (2000) Plant Physiol 122: 1003-1013). In another embodiment, the expression vector is a co-integrated vector (also called hybrid Ti plasmids). More expression vectors and methods of using them can be found in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830. Each of the references mentioned herein is incorporated by reference in its entirety.

Optionally, the nucleic acid sequence encoding the gene of interest is also operably linked to a plant 3′ non-translated region (3′ UTR). A plant 3′ non-translated sequence is not necessarily derived from a plant gene. For example, it can be a terminator sequence derived from viral or bacterium gene, or T-DNA. The 3′ non-translated regulatory DNA sequence can include from about 20 to 50, about 50 to 100, about 100 to 500, or about 500 to 1,000 nucleotide base pairs and may contain plant transcriptional and translational termination sequences in addition to a polyadenylation signal and any other regulatory signals capable of effecting mRNA processing or gene expression. Non-limiting examples of suitable 3′ non-translated sequences are the 3′ transcribed non-translated regions containing a polyadenylation signal from the nopaline synthase (NOS) gene of Agrobacterium tumefaciens (Bevan et al., 1983, Nucl. Acid Res., 11:369), or terminator for the T7 transcript from the octopine synthase gene of Agrobacterium tumefaciens.

In some embodiments, the expression cassettes or the expression vectors of the present disclosure further comprise nucleic acids encoding one or more selection markers. The selection marker can be a positive selectable marker, a negative selectable marker, or combination thereof. A “positive selectable marker gene” encodes a protein that allows growth on selective medium of cells that carry the marker gene, but not of cells that do not carry the marker gene. Selection is for cells that grow on the selective medium (showing acquisition of the marker) and is used to identify transformants. A common example is a drug-resistance marker such as NPT (neomycin phosphotransferase), whose gene product detoxifies kanamycin by phosphorylation and thus allows growth on media containing the drug. Other positive selectable marker genes for use in connection with the present disclosure include, but are not limited to, a Neo gene (Potrykus et al., 1985), which codes for kanamycin resistance and can be selected for using kanamycin, G418; a bar gene, which codes for bialaphos (basta) resistance; a mutant aroA gene, which encodes an altered EPSP synthase protein (Hinchee et al., 1988), thus conferring glyphosate resistance; a nitrilase gene such as bxn from Klebsiella ozaenae, which confers resistance to bromoxynil (Stalker et al., 1988); a mutant acetolactate synthase gene (ALS), which confers resistance to imidazolinone, sulfonylurea or other ALS inhibiting chemicals (European Patent Application 154,204,1985); a methotrexate resistant DHFR gene (Thillet et al., 1988), or a dalapon dehalogenase gene that confers resistance to the herbicide dalapon; the pat gene from Streptomyces viridochromogenes, which encodes the enzyme phosphinothricin acetyl transferase (PAT) and inactivates the active ingredient in the herbicide bialaphos, phosphinothricin (PPT); or a mutated anthranilate synthase gene that confers resistance to 5-methyl tryptophan. Additional positive selectable marker genes include those genes that provide resistance to environmental factors such as excess moisture, chilling, freezing, high temperature, salt, and oxidative stress. Of course, when it is desired to introduce such a trait into a plant as a “gene of interest”, the selectable marker cannot be one that provides for resistance to an environmental factor.

Markers useful in the practice of the claimed disclosure include: an “antifreeze” protein such as that of the winter flounder (Cutler et al., 1989) or synthetic gene derivatives thereof; genes which provide improved chilling tolerance, such as that conferred through increased expression of glycerol-3-phosphate acetyltransferase in chloroplasts (Murata et al., 1992; Wolter et al., 1992); resistance to oxidative stress conferred by expression of superoxide dismutase (Gupta et al., 1993), and may be improved by glutathione reductase (Bowler et al., 1992); genes providing “drought resistance” and “drought tolerance”, such as genes encoding for mannitol dehydrogenase (Lee and Saier, 1982) and trehalose-6-phosphate synthase (Kaasen et al., 1992).

A “negative selectable marker gene” encodes a protein that prevents the growth of a plant or plant cell on selective medium of plants that carry the marker gene, but not of plants that do not carry the marker gene. Selection of plants that grow on the selective medium provides for the identification of plants that have eliminated or evicted the selectable marker genes. An example is CodA (Escherichia coli cytosine deaminase), whose gene product deaminates 5-fluorocytosine (which is normally non-toxic as plants do not metabolize cytosine) to the toxic 5-fluorouracil. Other negative selectable markers include the haloalkane dehalogenase (dhlA) gene of Xanthobacter autotrophicus GJ10 which encodes a dehalogenase, which hydrolyzes dihaloalkanes, such as 1,2-dichloroethane (DCE), to a halogenated alcohol and an inorganic halide (Naested et al. (1999) Plant J. 18 (5):571-6). Each of the publications on selectable markers mentioned herein is incorporated by reference in its entirety.

In some embodiments, the expression vectors comprise border-like sequences. A border-like sequence can be isolated from a plant genome and be modified or mutated to change the efficiency by which it is capable of integrating a nucleotide sequence into another nucleotide sequence.

The present disclosure also provides methods for breeding strawberry plants which have at least one modified flowering time regulation gene. In some embodiments, the methods comprise (i) crossing any one of the plants of the present disclosure comprising a modified gene as a donor to a recipient plant to create a F1 population; (ii) evaluating the phenotypes in the offspring derived from said F1 population; and (iii) selecting offspring that have prolonged flowering time. In some embodiments, the recipient plant is an elite line having one or more certain agronomically important traits.

The most common method for the introduction of new genetic material into a plant genome involves the use of living cells of the bacterial pathogen Agrobacterium tumefaciens to literally inject a piece of DNA, called transfer or T-DNA, into individual plant cells (usually following wounding of the tissue) where it is targeted to the plant nucleus for chromosomal integration. There are numerous patents governing Agrobacterium mediated transformation and particular DNA delivery plasmids designed specifically for use with Agrobacterium, for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO09967357, U.S. Pat. No. 4,399,216, WO8303259, U.S. Pat No. 5,731,179, EP068730, WO9516031, U.S. Pat. No. 5,693,512, U.S. Pat. No. 6,051,757 and EP904362A1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with plant derived border sequences are also well known to those skilled in the art and can have applicability in the present disclosure. See, for example, U.S. Pat. No. 7,250,554, which is incorporated herein by reference in its entirety.

Nehra et al. (1990) Plant Cell Rep. 9:293-298) and James et al. (1990) Acta Horticulturae 280:495-502) describe methods for Agrobacterium-mediated transformation of strawberry either via callus or leaf disk regeneration system. Since then, further research on regeneration and transformation via Agrobacterium tumefaciens have been performed in different combinations of growth regulators and culture conditions using various strawberry cultivars since the success of transformation was cultivar-dependent. U.S. Pat. No. 6,274,791 describes methods for Agrobacterium-mediated transformation and regeneration of strawberry plants. See, for example, U.S. Pat. No. 6,274,791, which is incorporated herein by reference in its entirety.

Traditional methods for breeding strawberry plants can be utilized to create additional strawberry plants based on the present disclosure, such as those described in Strawberry; History, Breeding and Physiology by Darrow G M (1966), and U.S. Pat. No. 6,598,339 which is hereby incorporated by reference in its entirety. The cultivated strawberry (F.×ananassa) is an interspecific hybrid between the wild octaploid species F. chiloensis L. and F. virginiana Duch., which was first introduced in the 1750s (Darrow, 1966). Using recurrent mass selection, intraspecific and interspecific crosses have been utilized to make new cultivars. Nowadays, there are more than twenty Fragaria species possessing multiple ploidy that change in size, color, flavor, shape, degree of fertility, season of ripening, susceptibility to disease and constitution of plant (Biswas et al. (2009) Sci Hortic. 122:409-416). With intraspecific crosses of the cultivated strawberry variety (F.×ananassa), improved agronomic traits are introduced into new cultivars. Pedigree selection, crossing of the best genotypes, and further selection are used for breeding for new strawberry cultivars because the strawberry cultivars are heterozygous and sensitive to inbreeding. Strawberry cultivars are then vegetatively propagated through runners (or stolons) as clones (Hancock, 1999). Also, a new strawberry cultivar can be developed through the induction of somaclonal variation from in vitro tissue culture and selection of suitable variants for further cultivation (Biswas et al., 2009). Somaclonal variation occurs by changes in chromosome number (polyploidy) or chromosome rearrangements by insertions, deletions, translocations, or mutation. The success of plant breeding by somaclonal variation depends on the selection of genetically stable somaclones.

Classic breeding methods can be included in the present disclosure to introduce one or more modified gene of the present disclosure into other plant varieties, or other close-related species that are compatible to be crossed with the transgenic plant of the present disclosure.

A strawberry plant of the present disclosure has upregulated flowering cycle pathway even without being conditioned at high elevation. Such strawberry plants can be grown at low elevations, without the need for conditioning. In addition, such strawberry plants can be grown without the use of fumigant pesticides, such as methyl bromide. The present disclosure reduces the amount of land necessary to grow commercially appealing strawberries from plug plants while increasing yield, and reducing pest contamination in an environmentally safe capacity.

In some embodiments, genetically modified strawberry plants of the present disclosure can be grown directly in a production field without a prior growth season in a field where the plug plants are vernalized. By this method, the time necessary for plug plant production is reduced. In some embodiments, the time necessary for plug plant production is reduced by at least 0.5 month, 1 month, 2 months, 3 months, 4 months, 5 months, 5.5 months, 6 months, 6.5 months, 7 months, 8 months, or more. The disclosure method also reduces use of fumigant pesticides.

In some embodiments, methods of the present disclosure use strawberry plants with reduced expression of endogenous TFL gene(s), and these plants have increased yield when compared to control plants without the modified flowering time signaling pathway. In some embodiments, the methods increase fruit production of the plants. In some embodiments, the yield is measured by fruit weight produced per plant, or fruit number per plant, or per acre, within a given period of time, such as per season or per year. In some embodiments, the yield is increased by at least about 5%, about 10%, about 15%, about 20%, about 25%, about 30%, about 40%, about 50%, about 60%, about 70%, about 80%, about 90%, about one time, about two times, about three times, or more.

Strawberry plants of the present disclosure can be propagated in sterile-soil in greenhouses and/or screenhouses and can be planted in the fruiting field based on the weather condition. Also, the strawberry plants can be grown in the systematically controlled greenhouses and/or screenhouses all year long. The present disclosure allows continuous fruit production without any gaps throughout the year(s).

The present disclosure can be applied to other strawberry varieties to make them grow and produce fruits independent of environmental cues like photoperiod linked to day length and/or temperature (for example, vernalization).

Benefits of the present disclosure include, but are not limited to:

1. Helping growers to get access to pest-free nursery stock or plug plants that do not require conditioning.

2. Lowering the cost of labor, land, transportation, nutrients, fumigants of strawberry stock plant production involved in high-elevation nursery.

3. Saving lag time of about 6-6.5 months in which a stock plant can be provided to the grower or a distribution center, since it reduces the steps involved in stock plant production.

4. Reducing or eliminating the need to use fumigant pesticides.

5. Extending the growing season as in plants with reduced TFL1 expression, fruit product can start earlier (example, in Florida and Southern California when the fruit prices are the highest) or year-round in greenhouses.

6. Increasing fruit production (yield) throughout the season without gaps in fruit production.

7. Making any variety suitable for growth under long or short days and independent of temperature and affecting dynamics of strawberry varieties.

The present disclosure changes the industry practice of how strawberry nursery stock plants are produced and saves cost of transporting, planting, labor and land cost associated with high-elevation nursery. The method is more environmental friendly as it reduces the need for fumigant pesticides to control pests, pathogens and weeds.

Unless defined otherwise, all technical and scientific terms used herein have the same meaning as commonly understood by one of ordinary skill in the art to which this disclosure belongs. Generally, the nomenclature used herein, and the laboratory procedures in cell culture, molecular genetics, and nucleic acid chemistry and hybridization described herein, are those well-known and commonly employed in the art. Standard techniques are used for recombinant nucleic acid methods, polynucleotide synthesis, microbial culture, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. The techniques and procedures are generally performed according to conventional methodology disclosed, for example, in Molecular cloning a laboratory manual, 2ed., Cold Springs Harbor Laboratory Press, Cold Springs Harbor, N.Y. (1989), and Current protocols in molecular biology, John Wiley & Sons, Baltimore, Md. (1989).

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference. Nothing herein is to be construed as an admission that the present disclosure is not entitled to antedate such publication by virtue of prior disclosure.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

All publications, patents and patent applications, including any drawings and appendices, and all nucleic acid sequences and polypeptide sequences identified by GenBank Accession numbers, herein are incorporated by reference to the same extent as if each individual publication or patent application was specifically and individually indicated to be incorporated by reference.

The foregoing description includes information that may be useful in understanding the present disclosure. It is not an admission that any of the information provided herein is prior art or relevant to the presently claimed disclosures, or that any publication specifically or implicitly referenced is prior art.

### EXAMPLES

### Example 1

### Plant Transformation Vectors

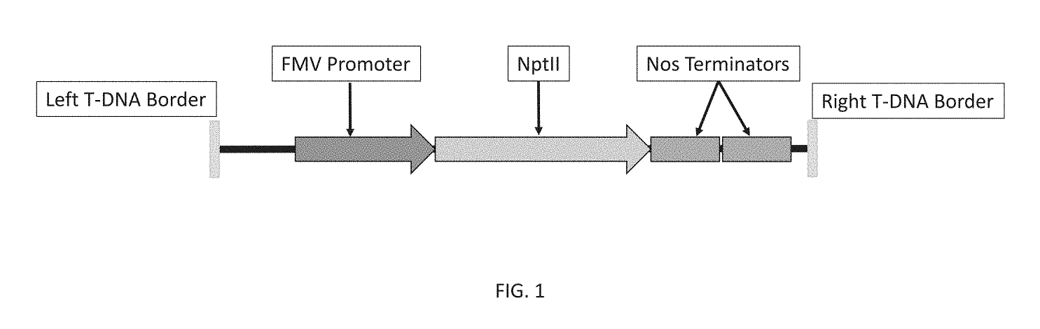

Plant transformation vectors were constructed using standard molecular biology techniques. The base vector is pSIM2402 and contains a plant transformation cassette comprising an NptII gene conferring resistance to antibiotics such as kanamycin, neomycin, genticin, and paromomycin. The insert DNA segment from the left T-DNA border to the right T-DNA border and containing the NptII gene cassette is 2304 bp in length and is presented as SEQ ID No: 13. The genetic elements of the pSIM2402 insert DNA are shown in Table 1, and illustrated in FIG. 1.

TABLE 1
Genetic Elements of pSIM2402 insert region (SEQ ID NO: 13)

| | | Accession | | Size | |
|---|---|---|---|---|---|
| Genetic Element | Origin | Number | Position | (bp) | Intended Function |
| 1. Left Border | pCambia-1301 | AF234297 | 1-26 | 26 | Secondary cleavage site releases |
| (LB) site | | | | | ssDNA insert from pSIM2402 (van |
| | | | | | Haaren et al., 1989; Hajdukiewicz |
| | | | | | et al. 1994) |
| 2. LB region | pCambia-1301 | AF234297 | 27-278 | 252 | Buffer for truncations during |
| | | | | | insertion |
| 3. Intervening | Synthetic | n/a | 279-303 | 25 | Sequence used as multiple cloning |
| Sequence | | | | | site for DNA cloning |
| 4. FMV promoter | pBI121 | HM047294 | 304-885 | 582 | Drives expression of the NPTII |
| | | | | | gene |
| 5. Intervening | Synthetic | n/a | 886-897 | 12 | Sequence used for DNA cloning |
| sequence | | | | | |
| 6. Neomycin | pWS32 | AF433043 | 898-1692 | 795 | Kanamycin resistance gene |
| phosphotransferase | | | | | |
| gene | | | | | |
| 7. Intervening | Synthetic | n/a | 1,693-1698 | 6 | Sequence used for DNA cloning |
| sequence | | | | | |
| 8. Nos terminator | pINDEX2 | AF294980 | 1,699-1974 | 276 | Terminates transcription of NPTII. |
| 9. Intervening | Synthetic | n/a | 1,975-2008 | 34 | Sequence used for DNA cloning |
| Sequence | | | | | |
| 10. Nos terminator | pCambia-1301 | AF234297 | 2,009-2,261 | 253 | Terminates transcription |
| 17. Right Border | pCambia-1301 | AF234297 | 2,262-2279 | 18 | Buffer for truncations during |
| (RB) region | | | | | insertion |
| 18. RB sequence | pCambia-1301 | AF234297 | 2,280-2,304 | 25 | Primary cleavage releases ssDNA |
| | | | | | insert from pSIM2402 (van Haaren |
| | | | | | et al., 1989) |

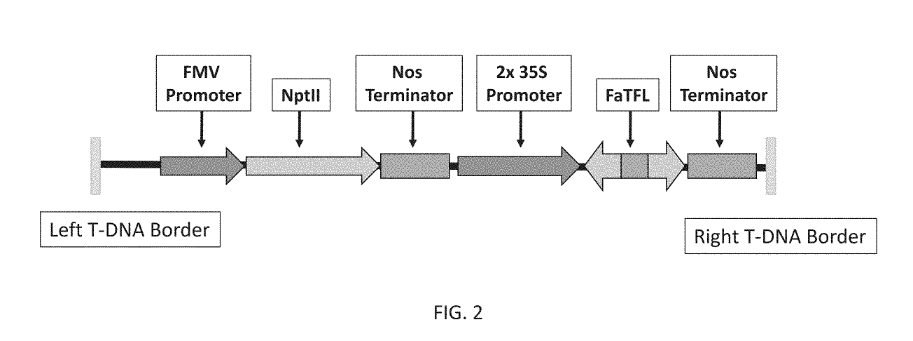

The transformation vector for reducing expression of TFL1 is pSIM2441 and contains (1) an expression cassette for expression of the NptII gene, and (2) an expression cassette for expression of the TFL1 gene-silencing cassette (FaTFL). The insert DNA segment from the left T-DNA border to the right T-DNA border of the pSIM2441 vector is 4307 bp in length and is presented as SEQ ID No: 14. The genetic elements of the pSIM2441 insert DNA are shown in Table 2, and illustrated in FIG. 2. The inverted repeat region of the FaTFL expression cassette comprises SEQ ID NO: 19, SEQ ID NO: 20, and SEQ ID NO: 21, operably linked as indicated in Table 2.

TABLE 2
Genetic Elements of pSIM2441 insert region (SEQ ID NO: 14)

| | | Accession | Position | Size | |
|---|---|---|---|---|---|
| Genetic Element | Origin | Number | (pSIM2441) | (bp) | Intended Function |
| 1. Left Border | pCambia-1301 | AF234297 | 1-26 | 26 | Secondary cleavage site |
| (LB) site | | | | | releases ssDNA insert from |
| | | | | | pSIM2402 (van Haaren et al., |
| | | | | | 1989; Hajdukiewicz et al. 1994) |
| 2. LB region | pCambia-1301 | AF234297 | 27-282 | 256 | Buffer for truncations during |
| | | | | | insertion |
| 3. Intervening | Synthetic | n/a | 283-307 | 25 | Sequence used as multiple |
| Sequence | | | | | cloning site for DNA cloning |
| 4. FMV promoter | pBI121 | HM047294 | 308-889 | 582 | Drives expression of the |
| | | | | | NPTII gene |
| 5. Intervening | Synthetic | n/a | 890-901 | 12 | Sequence used for DNA cloning |
| sequence | | | | | |
| 6. Neomycin | pWS32 | AF433043 | 902-1,696 | 795 | Kanamycin resistance gene |
| phosphotransferase | | | | | |
| gene | | | | | |
| 7. Intervening | Synthetic | n/a | 1,697-1,702 | 6 | Sequence used for DNA cloning |
| sequence | | | | | |
| 8. Nos terminator | pINDEX2 | AF294980 | 1,703-1,978 | 276 | Terminates transcription of |
| | | | | | NPTII. |
| 9. Intervening | Synthetic | n/a | 1,979-1994 | 16 | Sequence used for DNA cloning |
| Sequence | | | | | |
| 10. 35 s promoter | pCambia-1301 | AF234297 | 1,995-2,846 | 852 | Drives expression of the SAAT |
| | | | | | gene |
| 11. Intervening | Synthetic | n/a | 2,847-2,852 | 6 | Sequence used for DNA cloning |
| sequence | | | | | |
| 12. FaTFL like gene | F. x ananassa | JN788264 | 2,853-3,185 | 333 | Generates dsRNA to |
| fragment (anti- | | | | | downregulate TFL transcripts |
| sense orientation) | | | | | |
| SEQ ID NO: 19 | | | | | |
| 13. FaTFL gene | F. x ananassa | JN788264 | 3,186-3,368 | 183 | Sequence between the inverted |
| fragment (sense | | | | | repeat; transcript forms hairpin |
| orientation) | | | | | loop |
| SEQ ID NO: 20 | | | | | |
| 14. FaTFL gene | F. x ananassa | JN788264 | 3,369-3,701 | 333 | Generates dsRNA to |
| fragment (sense | | | | | downregulate TFL transcripts |
| orientation) | | | | | |
| SEQ ID NO: 21 | | | | | |
| 15. Intervening | Synthetic | n/a | 3,702-3,741 | 40 | Sequence used for DNA cloning |
| Sequence/linker | | | | | with multiple cloning sites |
| sequence | | | | | |
| 16. Nos Terminator | pCambia-1301 | AF234297 | 3,742-3,994 | 253 | Terminates transcription Inv1. |
| 17. Right Border | pCambia-1301 | AF234297 | 3,995-4,012 | 18 | Buffer for truncations during |
| (RB) region | | | | | insertion |
| 18. RB sequence | pCambia-1301 | AF234297 | 4,013-4,037 | 25 | Primary cleavage releases |
| | | | | | ssDNA insert from pSIM2441 |
| | | | | | (van Haaren et al., 1989) |

### Example 2

### Strawberry Plant Transformation

Strawberry plants (LF9 germplasm) were grown in Magenta boxes (Magenta™, Model GA-7, Millipore Sigma, St. Louis, Mo.) containing Fragaria ananassa rooting (FaR) medium (1/2-X M519 (PhytoTechnology Laboratories, Shawnee, Kans.), 15 g/L sucrose, 6 g/L agar, pH 5.7) inside a Percival growth chamber (Percival Scientific, Inc., Model CU-36L4, Perry, Iowa) with a 16 hour photoperiod at 23° C. Leaves of 4-6 week old plants were cut and placed on a filter paper moistened with sterile MS liquid medium (1X M519, 30 g/L sucrose, pH 5.7). The outer edges of each leaf were removed, and each leaf was cut into 3-5×3-5 mm rectangular explant sectors. Explants were placed onto pre-culture medium (Shoot regeneration media 4 (SRM4): 1X M519, 30 g/L sucrose, 6 g/L agar, pH 5.7, 2 mg/L thidiazuron, 0.5 mg/L indole-3-butyric acid) with the abaxial surface (under side) in contact with the medium. Explants on pre-culture SRM4 media were placed in a Percival growth chamber at 23° C. in the dark for 7 days.

After the pre-culture period, the explants were placed in a sterile petri plate with 15 to 25 ml of a transformed Agrobacterium culture containing 200 μM acetosyringone, and 0.4% Tween-20. The petri plates were sealed with Parafilm, and incubated in the dark for 30 minutes at room temperature with gentle agitation on an orbital shaker. The transformed Agrobacterium strain GV3101 had been transformed with either the pSIM2402 vector or the pSIM2441 vector, and a culture grown to an Optical Density (OD600 nm) of 0.3. At the end of the incubation, the explants were transferred to sterile filter paper to remove excess Agrobacterium and placed on co-culture medium (SRM4 media) without selection. These explants were placed in a Percival growth chamber at 23° C. in the dark for 48 hours.

After the co-culture period, explants were transferred to recovery shoot regeneration media (SRM4) with 150 mg/L Timentin, 1.2 ml Plant Preservative Mixture (PPM), 50 mg/L Validamycin A, and 5 mg/L kanamycin for plant selection. These explants were placed in a Percival growth chamber at 23° C. in the dark for 7 days.

After the recovery period, explants were transferred to selection shoot regeneration medium (SRM4) with 150 mg/L Timentin, 1.2 ml PPM, and 25 mg/L kanamycin. These explants were placed in a Percival growth chamber with a 16 hour photoperiod at 23° C. The explants were subcultured onto new selection SRM4 media every 4 weeks until shoot clumps of at least 4-5 mm in length were achieved.

As shoot clumps formed, they were cut from the calli (1-2 independent shoot clumps per explant) and transferred to Magenta boxes containing FaR media with 150 mg/L Timentin and 1.2 ml PPM along with 75 mg/L kanamycin. Shoot clumps in Magenta boxes were placed in a Percival growth chamber with a 16 hour photoperiod at 23° C. Shoot clumps were subcultured onto new FaR media with 150 mg/L Timentin and 1.2 ml PPM along with 75 mg/L kanamycin every 4 weeks for two additional subcultures (2nd and 3rd subcultures). After 4 weeks on the 3rd subculture, individual rooted shoots were collected onto FaR media with 150 mg/L Timentin and 1.2 mL PPM for 14 days.

Tissue samples were collected from the 14-day shoots, and used for PCR analysis. DNA was extracted from the tissue samples using a high-throughput DNA extraction method (Xin et al. (2003) Biotechniques 34:820-826). For the analysis of shoots regenerated from transformation with pSIM2402, the PCR primers amplified a 501 bp fragment spanning the region from position 678 to 1178 of SEQ ID NO: 13. For the analysis of shoots regenerated from transformation with pSIM2441, the PCR primers amplified a 652 bp fragment spanning the region from position 2,711 to 3,362 of SEQ ID NO: 14. Table 3 lists the PCR primers and primer sequences for screening plants regenerated from transformation with pSIM2402 or pSIM2441 vectors.

TABLE 3
PCR Primers for screening plants transformed
with pSIM2402 or pSIM2441

| | | SEQ | |
|---|---|---|---|
| Vector | Primer name | ID NO: | Primer Sequence |
| pSIM2402 | RF84-FMVPCR 1F | 17 | cgcacctaccaaaagcatct |
| | RF85-KANPCR 1R | 18 | caatagcagccagtcccttc |
| pSIM2441 | RF161-2441 1F | 15 | caaccacgtcttcaaagcaa |
| | RF162-2441 1R | 16 | catgtcgcctccttgaatct |

For each of the two vectors pSIM2402 and pSIM2441, Table 4 shows the number of explants cut and used for transformation, the number of rooted shoots, and the number of PCR positive plants. All of the PCR positive plants were transferred to soil-less growth medium in 6-inch pots and moved to a greenhouse.

TABLE 4
Scoring of LF9 plants transformed with pSIM2402 or pSIM2441

| | # of Explants | # of Rooted | # of PCR Positive |
|---|---|---|---|
| Vector | Cut | Shoots | Plants |
| pSIM2402 | 200 | 47 | 22 |
| pSIM2441 | 208 | 42 | 33 |

### Example 3

### Greenhouse Assessment

Strawberry plants regenerated from transformation with pSIM2402 or pSIM2441 vectors and scored as positive by the PCR assay were transferred to soil-less growing medium in 6-inch pots, and grown in a greenhouse. The greenhouse ambient temperatures were maintained at 60-65° F. during the day and 50-55° F. during the night. Light was provided from 6 AM to 10 PM, which constitutes a long day photoperiod. Natural light was the main light source, with high-pressure sodium fixtures providing supplemental lighting as needed. Plants were hand watered as necessary, and fertilized every two weeks. The lines developed from transformation with pSIM2441 vector (with downregulation of FaTFL) initiated flower buds 8 weeks after planting. The lines developed from transformation with the control vector pSIM2402 initiated buds 17 weeks after planting. At 20 weeks post planting, fruit and bud formation was determined for wild-type (WT) LF9 plants (8 separate plants), plants transformed with pSIM2402 (19 independent lines or events), and plants transformed with pSIM2441 (29 independent lines or events). Table 5 shows the number of genetic events evaluated, and the percentage of the plants producing fruit and/or buds. These results demonstrate that plants transformed with pSIM2441 initiated flowering 9 weeks earlier than plants transformed with the control vector pSIM2402. Additionally, the results show that plants transformed with pSIM2441 had an increase in flower and/or fruit production at 20 weeks after planting compared to either WT or control transformed plants.

TABLE 5
Greenhouse assessment of fruit or bud
formation 20 weeks after planting

| Vector | No. of Genetic Events | % of Plants Producing Fruit or Buds |
|---|---|---|
| WT | 8 | 37.5 |
| pSIM2402 | 19 | 5 |
| pSIM2441 | 29 | 93 |

### Example 4

### First Year Field Assessment

Bare root daughter plants propagated from the plants grown in the greenhouse, were shipped to a low elevation site, and planted into pots for rooting. The potted daughter plants were maintained in a screenhouse, and watered and fertilized as necessary. Once rooted in the pots, one replicate per genetic event was planted into the ground two months later in central California. Events were randomized within the trial. In this trial, there were 25 WT plants, 26 events from transformation with control vector pSIM2402, and 25 events from transformation with vector pSIM2441. Six weeks after planting into the ground, all inflorescences were removed to synchronize flower production. Inflorescence counts were documented at weeks 8, 12, 16, and 20 after planting. An inflorescence was counted if at least one flower reached anthesis. The number of inflorescences were counted then removed each assessment date. The total number of inflorescence per plant counted at weeks 8, 12, 16, and 20 were summed, and the total number of inflorescence per plant is presented in Table 6. The results show that the WT and the events from control vector pSIM2402 gave essentially the same number of total inflorescence per plant (see Table 6). In contrast, the events from transformation vector pSIM2441 had an average total number of inflorescence per plant that was about double that of WT and control pSIM2402 events (see Table 6). The total number of inflorescence over the period 8-20 weeks after planting for 25 individual pSIM2441 events ranged from a low of 4 (Event 2441-15) to a high of 42 (Event 2441-37) (see Table 7).

TABLE 6
First year field trial scoring total number
of inflorescence 8-20 weeks after planting

| Vector | No. of Events | Avg. Total No. Inflorescence |
|---|---|---|
| WT | 25 | 12.32 |
| pSIM2402 | 26 | 12.27 |
| pSIM2441 | 25 | 23.80 |

TABLE 7
First year field trial scoring total number of inflorescence
8-20 weeks after planting for 25 individual events
from transformation with pSIM2441

| Event ID | Total No. Inflorescence |
|---|---|
| 2441-01 | 10 |
| 2441-02 | 25 |
| 2441-03 | 22 |
| 2441-06 | 35 |
| 2441-08 | 24 |
| 2441-09 | 15 |
| 2441-10 | 7 |
| 2441-11 | 32 |
| 2441-15 | 4 |
| 2441-16 | 18 |
| 2441-17 | 20 |
| 2441-18 | 29 |
| 2441-20 | 25 |
| 2441-21 | 51 |
| 2441-25 | 33 |
| 2441-26 | 19 |
| 2441-28 | 16 |
| 2441-29 | 16 |
| 2441-30 | 16 |
| 2441-31 | 15 |
| 2441-32 | 21 |
| 2441-33 | 37 |
| 2441-35 | 22 |
| 2441-37 | 42 |
| 2441-39 | 41 |

### Example 5

### Second Year Field Assessment

Bare root daughter plants were harvested and shipped from both a low elevation nursery (Central California), and a high elevation nursery (Northern California) and planted into a production field. The low elevation nursery plants did not receive chilling (Non-Chilled source plants), while the high elevation nursery plants received chill hours similar to standard practices for a commercial variety (Chilled source plants). Daughter plants were planted into a traditional fruit production area according to standard commercial practices on Nov. 11, 2015. At planting, there was one plot per genetic event, with each plot comprising 12 plants. For WT plants, there were 6 plots of 12 plants each. There were 6 individual genetic events, 1 plot per event, evaluated for events from pSIM2402. There were 10 individual genetic events, 1 plot per event, evaluated for events from pSIM2441. The plots were not randomized within the trial. The number of plants per plot with at least one flower that had reached anthesis was documented weekly starting at week 1 through week 16. Plots were thinned to 10 plants per plot in mid-March, before yield assessments began. Total yield assessments began on Apr. 7, 2016 and ended on Aug. 29, 2016, with fruit harvested every 2-3 days over the period, counted and weighted.

For chilled source plants, the number of plants reaching anthesis at 12 weeks post planting was about equal for WT and pSIM2402 plants (24 and 29 total plants, respectively). In contrast, there was a 2 to 3-fold increase in the total number of pSIM2441 plants reaching anthesis (74 plants) (see Table 8). The number of pSIM2441 plants reaching anthesis for individual genetic events ranged from 5 to 11, see Table 9. These results indicate that some strawberry events generated by transformation with pSIM2441 leads to increased anthesis in chilled source plants.

TABLE 8
Number of plants at anthesis for Chilled
Source Plants at 12 weeks post planting

| | Total No. of | No. of Plants at | % of Plants at |
|---|---|---|---|
| Vector | Established Plants | Anthesis | Anthesis |
| WT | 72 | 24 | 33% |
| pSIM2402 | 71 | 29 | 41% |
| pIM2441 | 118 | 74 | 63% |

TABLE 9
Independent genetic pSIM2441 plants scored for anthesis
at 12 weeks post planting, Chilled Source Plants.

| | Total No. of | No. of Plants at | % of Plants at |
|---|---|---|---|
| Event ID | Established Plants | Anthesis | Anthesis |
| 2441-2 | 12 | 7 | 58% |
| 2441-8 | 12 | 9 | 75% |
| 2441-21 | 11 | 5 | 45% |
| 2441-25 | 12 | 8 | 67% |
| 2441-26 | 12 | 11 | 92% |
| 2441-32 | 12 | 9 | 75% |
| 2441-33 | 12 | 9 | 75% |
| 2441-35 | 12 | 6 | 50% |
| 2441-37 | 11 | 5 | 45% |
| 2441-39 | 12 | 5 | 42% |

For non-chilled source plants, the number of plants reaching anthesis at 12 weeks post planting was 4 for WT plants, 1 for pSIM2402 plants, and 37 for pSIM2441 plants (see Table 10). These numbers represent 6% (WT), 1% (pSIM2402), and 31% (pSIM2441) of the total number of established plants. The number of pSIM2441 plants reaching anthesis for individual genetic events ranged from a low of 1 plant, or 8% of the number of established plants (Event ID 2441-35), to a high of 7 plants, or 58% of the number of established plants (Event ID 2441-33) (see Table 11). These results indicate that some strawberry events generated by transformation with pSIM2441 led to increased anthesis in non-chilled source plants.

TABLE 10
Number of plants at anthesis for Non-Chilled
Source Plants at 12 weeks post planting

| | Total Number of | No. of Plants at | % of Plants at |
|---|---|---|---|
| Vector | Established Plants | Anthesis | Anthesis |
| WT | 72 | 4 | 6% |
| pSIM2402 | 72 | 1 | 1% |
| pSIM2441 | 120 | 37 | 31% |

TABLE 11
Independent genetic pSIM2441 plants scored for anthesis
at 12 weeks post planting, Non-Chilled Source Plants.

| | Total No. of | No. of Plants at | % of Plants |
|---|---|---|---|
| Event ID | Established Plants | Anthesis | Anthesis |
| 2441-2 | 12 | 2 | 17% |
| 2441-8 | 12 | 2 | 17% |
| 2441-21 | 12 | 6 | 50% |
| 2441-25 | 12 | 5 | 42% |
| 2441-26 | 12 | 4 | 33% |
| 2441-32 | 12 | 5 | 42% |
| 2441-33 | 12 | 7 | 58% |
| 2441-35 | 12 | 1 | 8% |
| 2441-37 | 12 | 2 | 17% |
| 2441-39 | 12 | 3 | 25% |

For chilled source plants, the average total yield at 41 weeks post planting was 17,219 grams for WT; 15,848 grams for pSIM2402 plants; and 18,967 grams for pSIM2441 plants, see Table 12. The average total yield for individual genetic events of pSIM2441 plants ranged from a low of 8,360 grams (Event ID 2441-21) to a high of 24,945 grams (Event ID 2441-25) (see Table 13). These results indicate that some strawberry events generated by transformation with pSIM2441 led to increased average total yield in chilled source plants at 41 weeks post planting.

TABLE 12
Average total yield for Chilled Source
Plants at 41 weeks after planting

| Vector | Avg. Total Weight in Grams |
|---|---|
| WT | 17219 |
| pSIM2402 | 15848 |
| pSIM2441 | 18967 |

TABLE 13
Average total yield for Chilled Source Plants at 41
weeks after planting for independent pSIM2441 events

| Event ID | Avg. Total Weight in Grams |
|---|---|
| 2441-2 | 23515 |
| 2441-8 | 16870 |
| 2441-21 | 8360 |
| 2441-25 | 24945 |
| 2441-26 | 20990 |
| 2441-32 | 21580 |
| 2441-33 | 19220 |
| 2441-35 | 18175 |
| 2441-37 | 17400 |
| 2441-39 | 18620 |

For non-chilled source plants, the average total yield at 41 weeks post planting was 14,795 grams for WT; 14,152 grams for pSIM2402 plants; and 20,354 grams for pSIM2441 plants, see Table 14. The average total yield for individual genetic events of pSIM2441 plants ranged from a low of 11,105 grams (Event ID 2441-21) to a high of 24,085 grams (Event ID 2441-2) (see Table 15). These results indicate that some strawberry events generated by transformation with pSIM2441 led to increased average total yield in NON-chilled source plants at 41 weeks post planting.

TABLE 14
Average Total Yield Non-Chilled Source
Plants at 41 weeks after planting

| Vector | Average Total Weight in Grams |
|---|---|
| WT | 14795 |
| pSIM2402 | 14152 |
| pSIM2441 | 20354 |

TABLE 15
Average Total Yield Non-Chilled Source Plants at 41
weeks after planting for independent pSIM2441 events

| Event ID | Average Total Weight in Grams |
|---|---|
| 2441-2 | 24085 |
| 2441-8 | 20275 |
| 2441-21 | 11105 |
| 2441-25 | 23015 |
| 2441-26 | 21485 |
| 2441-32 | 23295 |
| 2441-33 | 21640 |
| 2441-35 | 15905 |
| 2441-37 | 21400 |
| 2441-39 | 21339 |

### Example 6

### Third Year Field Assessment

Bare root daughter plants from a high elevation nursery (Northern California) were planted into a traditional fruit production area according to standard commercial practices on Nov. 10, 2016. At planting, there were four plots per genetic event, with each plot comprising 12 plants (chilled source plants from the high elevation nursery). There were 5 individual genetic events evaluated for pSIM2402 plants, and there were 9 individual genetic events evaluated for pSIM2441 plants. The plots were randomized within the trial. The number of plants per plot with at least one flower that had reached anthesis was documented weekly starting at week 1 thru week 19. The plots were thinned to 10 plants per plot in early April, before yield assessments began. Total yield assessments began on Apr. 11, 2017 and will continue thru September 2017, with fruit harvested every 2-3 days throughout the period, counted and weighted.

For the chilled source plants in the third year field trial, the number of plants reaching anthesis at 19 weeks post planting was 123 for WT plants, 112 for pSIM2402 plants, and 296 for pSIM2441 plants (see Table 16). These numbers represent 52% (WT), 47% (pSIM2402), and 84% (pSIM2441) of the total number of established plants. The number of pSIM2441 plants reaching anthesis for individual genetic events ranged from a low of 11 plants, or 46% of the number of established plants (Event ID 2441-35), to a high of 45 plants, or 96% of the number of established plants (Event ID 2441-26) (see Table 17). These results indicate that some strawberry events generated by transformation with pSIM2441 led to increased anthesis in chilled source plants.

TABLE 16
Third year field trial, chilled source plants, the total
number of plants at anthesis at 19 weeks post planting

| | Total No. of | No. of Plants at | % of Plants at |
|---|---|---|---|
| Vector | Established Plants | Anthesis | Anthesis |
| WT | 235 | 123 | 52 |
| pSIM2402 | 239 | 112 | 47 |
| pSIM2441 | 350 | 296 | 84 |

TABLE 17
Third year field trial chilled source plants, the number of plants at
anthesis at 19 weeks post planting for individual pSIM2441 events.

| | Total No. of | No. of Plants at | % of Plants at |
|---|---|---|---|
| Event ID | Established Plants | Anthesis | Anthesis |
| 2441-2 | 24 | 22 | 92% |
| 2441-8 | 43 | 41 | 95% |
| 2441-25 | 47 | 42 | 89% |
| 2441-26 | 47 | 45 | 96% |
| 2441-32 | 48 | 43 | 90% |
| 2441-33 | 23 | 15 | 65% |
| 2441-35 | 24 | 11 | 46% |
| 2441-37 | 48 | 38 | 79% |
| 2441-39 | 46 | 39 | 85% |

For the third year field trial with chilled source plants, the average total yield at 34 weeks post planting was 1,002 grams for WT; 1,079 grams for pSIM2402 plants; and 1,127 grams for pSIM2441 plants, see Table 18. The average total yield for individual genetic events of pSIM2441 plants ranged from a low of 988 grams (Event ID 2441-2) to a high of 1449 grams (Event ID 2441-32) for 34 weeks post planting (see Table 19). These results indicate that some strawberry events generated by transformation with pSIM2441 led to increased average total yield in chilled source plants at ## weeks post planting.

TABLE 18
Third year field trial average yield in grams
collected over the 34 weeks of the trial.

| Vector | Average Total Weight in Grams |
|---|---|
| WT | 1002 |
| pSIM2402 | 1079 |
| pSIM2441 | 1127 |

TABLE 19
Third year field trial average yield in grams for individual
pSIM2441 events collected over the 34 weeks of the trial

| Event ID | Average Total Weight in Grams |
|---|---|
| 2441-2 | 988 |
| 2441-8 | 1024 |
| 2441-25 | 1257 |
| 2441-26 | 1007 |
| 2441-32 | 1449 |
| 2441-33 | 1216 |
| 2441-35 | 1025 |
| 2441-37 | 1128 |
| 2441-39 | 1045 |

What is claimed is:

1. A method for growing a genetically modified plug plant with reduced activity of a transcription factor encoded by a TERMINAL FLOWER (TFL) gene, comprising growing the plug plant in or on a substrate in a nursery location, wherein the genetically modified plug plant flowers independently of temperature or photoperiod.

2. The method of claim 1, wherein the genetically modified plug plant belongs to the genus Fragaria.

3. The method of claim 2, wherein the genetically modified plug plant is a hybrid plant or a cultivar of the genus Fragaria.

4. The method of claim 3, wherein the genetically modified plug plant is Fragaria vesca, or Fragaria×ananassa.

5. The method of claim 3, wherein the genetically modified plug plant is a diploid, a tetraploid, a pentaploid, a hexaploid, a octoploid, a decaploid, or has an uncharacterized ploidy.

6. The method of claim 1, wherein the genetically modified plug plant is a strawberry plant.

7. The method of claim 6, wherein the genetically modified plug plant is a June-bearing strawberry plant, an early season June-bearing strawberry plant, an early midseason June-bearing strawberry plant, a midseason June-bearing strawberry plant, a late midseason June-bearing strawberry plant, a late season June-bearing strawberry plant, a short-day strawberry plant variety, a seasonal flowering strawberry plant variety, a long-day strawberry plant variety, a day-neutral strawberry plant variety, a perpetual flowering strawberry plant variety, a recurrent strawberry plant variety, a remontant strawberry plant variety, a long-day strawberry plant variety, or an everbearing strawberry plant variety.

8. The method of claim 1, wherein the nursery location is (1) a low elevation, (2) a high elevation, (3) a northern latitude for northern hemisphere commercial fruit production, (3) a southern latitude for southern hemisphere commercial fruit production, or (4) in a controlled environment facility.

9. The method of claim 1, wherein the yield of strawberry fruit from the genetically modified plug plants with reduced activity of the transcription factor encoded by the TFL gene is greater than the yield of strawberry fruit from non-genetically modified plug plants when grown under the same conditions.

10. The method of claim 1, wherein the substrate for growing a genetically modified plug plant with reduced activity of the transcription factor encoded by the TFL gene is not treated with a fumigant pesticide.

11. The method of claim 10, wherein the fumigant pesticide is methyl bromide, 1,3-dichloropropene, trichloronitromethane, chloropicrin, methyl iodide, tetrahydro-3,5-dimethyl-2 H-1,3,5-thiadiazine-2-thione, sodium N-m ethyl dithiocarbamate, potassium N-methyl dithiocarbamate, or a combination thereof.

12. The method of claim 1, wherein the TFL gene is a TFL1 gene.

13. The method of claim 12, wherein the TFL1 gene comprises a nucleotide sequence of SEQ ID NO:1, SEQ ID NO:4, SEQ ID NO:2, SEQ ID NO:5, SEQ ID NO:7, or a nucleotide sequence encoding a protein sequence of SEQ ID NO:3, SEQ ID NO:6, or SEQ ID NO:8.

14. The method of claim 12, wherein the reduced activity of the TFL1 gene is achieved by a method selected from the group comprising: reduced gene expression level, reduced gene copy number, reduced gene amplification, reduced RNA activity level, reduced mRNA abundance, reduced mRNA synthesis rate, reduced mRNA stability, reduced protein activity level, reduced protein synthesis, reduced protein abundance, reduced protein stability, reduced protein enzymatic activity, reduced protein phosphorylation, or a combination thereof.

15. The method of claim 14, wherein the reduced activity of the TFL1 gene is induced by RNA interference (RNAi), genome editing, or mutation of the endogenous TFL1 gene.

16. The method of claim 15, wherein the RNA interference is induced by expression in a cell of the Fragaria plant an RNAi cassette targeting the endogenous TFL1 gene, or by topical application of RNAi triggers targeting the endogenous TFL1 gene.

17. The method of claim 14, wherein the genome editing is by expression in a cell of the Fragaria plant a zinc-finger nuclease, a TALE-mediated nuclease, or an RNA-guided nuclease.

18. The method of claim 14, wherein mutation of the endogenous TFL1 gene is by chemical mutagenesis, radiation mutagenesis, transposon mutagenesis, insertional mutagenesis, signature tagged mutagenesis, site-directed mutagenesis, and/or natural mutagenesis.

19. The method of claim 11, wherein the genetically modified plug plant with reduced activity of the transcription factor TFL is a long-day variety, a short-day variety, or a day neutral variety.

20. A method to reduce activity of an endogenous TFL1 gene in a Fragaria plant, comprising transforming the Fragaria plant with the pSIM2441 vector.

21. The method of claim 20, wherein the pSIM2441 vector comprises the insert region of nucleotide sequence SEQ ID NO: 14.

22. The method of claim 21, wherein the insert region comprises an expression cassette, wherein the expression cassette comprises on the same strand in operable linkage a sense copy and an antisense copy of a fragment of the TFL1 gene, wherein the sense copy and the antisense copy of the fragment have a length sufficient for gene silencing when expressed in a cell of a transformed Fragaria plant, and wherein the expression cassette comprises SEQ ID NO: 19, SEQ ID NO: 20, and SEQ ID NO: 21, operably linked together.

23. A Fragaria plant or plug plant transformed with the pSIM2441 vector, wherein the pSIM2441 vector comprises the insert region of nucleotide sequence SEQ ID NO: 14.

24. The transformed Fragaria plant of claim 23, wherein the expression cassette when expressed in a cell of the Fragaria plant causes reduced activity of the endogenous TFL1.
